(19) United States
(12) Patent Application Publication
(10) Pub. No.: US 2016/0033531 A1
(43) Pub. Date: Feb. 4, 2016

(54) METHOD AND COMPOSITIONS FOR DETECTING BINDING TO THE PREGNANE X RECEPTOR

(71) Applicant: ST. JUDE CHILDREN'S RESEARCH HOSPITAL, Memphis, TN (US)

(72) Inventors: Taosheng Chen, Memphis, TN (US); Wenwei Lin, Memphis, TN (US)

(21) Appl. No.: 14/773,901

(22) PCT Filed: Mar. 15, 2014
(86) PCT No.: PCT/US14/29924

§ 371 Date: Sep. 9, 2015

Related U.S. Application Data
(60) Provisional application No. 61/786,483, filed on Mar. 15, 2013.

(51) Int. Cl. G01N 33/68; G01N 21/64

(52) U.S. Cl. CPC G01N 33/6875; G01N 21/6428; G01N 2333/70567; G01N 2021/6439; G01N 2500/04; G01N 2500/20

(57) ABSTRACT

In one aspect, the invention relates to compounds useful as fluorescence assay probes, methods of making same, and methods of using same to assay ligand binding interactions with PXR. In various aspects, the invention pertains to compositions comprising a polypeptide comprising a pregnane X receptor polypeptide, or a ligand binding fragment polypeptide thereof; and a molecule comprising a BODIPY residue and a vinca alkaloid residue. This abstract is intended as a scanning tool for purposes of searching in the particular art and is not intended to be limiting of the present invention.

### CROSS-REFERENCE TO RELATED APPLICATIONS

This Application claims the benefit of U.S. Provisional Application No. 61/786,483, filed on Mar. 15, 2013, which is incorporated herein by reference in its entirety.

### BACKGROUND

The pregnane X receptor (PXR) regulates the metabolism and excretion of xenobiotics and endobiotics by regulating the expression of drug-metabolizing enzymes and drug transporters. By affecting drug metabolism, changes in the expression of such mediators can influence the therapeutic and toxicologic response to drugs and cause adverse drug-drug interactions. The activity of PXR is largely regulated by direct ligand binding, and the unique structure of PXR allows the binding of a variety of drugs and prospective drugs. That is, a drug or prospective drug molecule can directly modulate the activity of PXR. As such, PXR is associated with multiple undesired drug-drug interactions.

Therefore, it is important to be able to evaluate whether or not drugs and prospective drug compounds bind to PXR. A variety of ligand-binding assay types can be applied to assessing binding interactions of a compound and PXR, including both radioactive and non-radioactive assay methodologies. Radioisotope-based binding assays (scintillation proximity assays) were initially used to investigate direct binding of PXR to its ligands. They assessed the competitive binding of a group of tritium-labeled putative PXR ligands, such as SR12813, TO901317, and NMTB, to PXR. Although radioactive assays can provide valuable data, they are not preferred due to the requirements for handling radioactive waste. Moreover, radioactive assays are poorly suited to high-throughput screening methods. Currently in drug discovery, fluorescence-based assays are preferred because of their sensitivity, non-radioactive nature, and ready availability of necessary instrumentation.

Typical fluorescence assay methods for determining ligand binding interactions include fluorescence resonance energy transfer (“FRET”) and time-resolved fluorescence energy transfer (“TR-FRET”). Both fluorescence assay methods require a donor (including a molecule labeled with such a fluorescent donor) and acceptor (including a molecule labeled with such a fluorescent acceptor) fluorescent molecule that can interact appropriately with the assay target. Typically, in a ligand binding fluorescence assay, the acceptor fluorescence molecule binds to the ligand binding site and is displaced by the test molecule, thus decreasing fluorescence energy transfer. In either assay modality, FRET or TR-FRET, the value of the data obtained is greatly increased when the structure of the donor and acceptor molecules are known. In particular, in order to properly assess the results the structure of the acceptor molecule which is displaced at the ligand binding site should be known.

Despite advances in ligand binding assay methods for PXR, there remains a need for sensitive fluorescence-based assays utilizing reporter molecules of known chemical structure. These needs and other needs are satisfied by the present invention.

### SUMMARY

In accordance with the purpose(s) of the invention, as embodied and broadly described herein, the invention, in one aspect, relates to compounds useful as fluorescence assay probes, methods of making same, and methods of using same to assay ligand binding interactions with PXR.

Disclosed are compositions comprising a polypeptide comprising a pregnane X receptor polypeptide, or a ligand binding fragment polypeptide thereof; and a molecule comprising a BODIPY residue and a vinca alkaloid residue.

Also disclosed are methods for identifying a test compound that binds to pregnane X receptor, the method comprising the steps of: (a) providing a solution comprising: i) a test compound; ii) a chimeric polypeptide comprising a pregnane X receptor polypeptide, or a ligand binding fragment polypeptide thereof, and a heterologous polypeptide; iii) a fluorescent donor molecule comprising chelated terbium and one or more moieties capable of binding the heterologous polypeptide; and iv) a fluorescent acceptor molecule comprising a BODIPY residue and a vinca alkaloid residue; (b) illuminating the solution, thereby causing fluorescence in the fluorescent donor molecule; and (c) measuring fluorescence emission from the fluorescent donor molecule and the fluorescent acceptor molecule.

Also disclosed are kits for measuring the binding activity of a test compound to a pregnane X receptor polypeptide, comprising: (a) a chimeric polypeptide comprising a pregnane X receptor polypeptide, or a ligand binding fragment polypeptide thereof, and a heterologous polypeptide; (b) a fluorescent donor molecule comprising chelated terbium and one or more moieties capable of binding the heterologous polypeptide; and (c) a fluorescent acceptor molecule comprising a BODIPY residue and a vinca alkaloid residue; and optionally, one or more of: (d) a compound known to bind pregnane X receptor; (e) instructions for measuring a fluorescence emission ratio of the fluorescent acceptor molecule to the fluorescent donor molecule in the presence of a test compound; and (f) instructions for determining the Kᵢ or IC₅₀ of a test compound from a fluorescence emission ratio of the fluorescent acceptor molecule to the fluorescent donor molecule in the presence of a test compound.

Also disclosed are kits for measuring the binding activity of a test compound to a pregnane X receptor polypeptide, comprising one or more of: (a) a chimeric polypeptide comprising a pregnane X receptor polypeptide, or a ligand binding fragment polypeptide thereof, and a heterologous polypeptide; (b) a fluorescent donor molecule comprising chelated terbium and one or more moieties capable of binding the heterologous polypeptide; and (c) a fluorescent acceptor molecule comprising a BODIPY residue and a vinca alkaloid residue; and comprising one or more of: (d) a compound known to bind pregnane X receptor; (e) instructions for measuring a fluorescence emission ratio of the fluorescent acceptor molecule to the fluorescent donor molecule in the presence of a test compound; and (f) instructions for determining the Kᵢ or IC₅₀ of a test compound from a fluorescence emission ratio of the fluorescent acceptor molecule to the fluorescent donor molecule in the presence of a test compound.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows representative data for the interaction of BODIPY Fl-vinblastine with GST-hPXR-LBD and Tb-anti-GST in the presence of vehicle control (DMSO; representing total binding), a high affinity binding ligand of hPXR-LBD (TO901317; representing non-specific binding), or subtraction of non-specific binding from total binding. Assay was performed using Invitrogen assay buffer.

FIG. 2 shows representative data for the interaction of BODIPY Fl-vinblastine with GST-hPXR-LBD and Tb-anti-GST. (Panel A) Interaction in the presence of vehicle control (DMSO) in the presence and absence of GST-hPXR-LBD or in the presence of different concentrations of a high affinity binding ligand of hPXR-LBD (TO901317). (Panel B) Signal-to-background ratio (signal/background) of BODIPY Fl-vinblastine interaction with GST-hPXR-LBD and Tb-anti-GST.

FIG. 3 shows representative data pertaining to the longitudinal signal stability of the interaction of BODIPY Fl-vinblastine with GST-hPXR-LBD and Tb-anti-GST. (Panel A) Data obtained for the interaction BODIPY Fl-vinblastine with GST-hPXR-LBD and Tb-anti-GST obtained at different times as indicated in the presence of either vehicle control (DMSO) or a high affinity binding ligand of hPXR-LBD (TO901317). (Panel B) Signal-to-background ratio (signal/background) from the data shown in Panel A. (Panel C) Z′-factor values of the interaction of BODIPY Fl-vinblastine with GST-hPXR-LBD and Tb-anti-GST at different times as indicated. (Panel D) Dose response curves for a high affinity binding ligand of hPXR-LBD (TO901317) in the presence of BODIPY Fl-vinblastine, GST-hPXR-LBD, and Tb-anti-GST at different times as indicated.

FIG. 4 shows representative data for the interaction of BODIPY Fl-vinblastine with GST-hPXR-LBD and Tb-anti-GST. (Panel A) Different concentrations of DMSO (as indicated) were assessed for the effect on the interaction of BODIPY Fl-vinblastine with GST-hPXR-LBD and Tb-anti-GST. (Panel B) Signal-to-background ratio (signal/background) from the data shown in Panel A. (Panel C) Dose response curves for a high affinity binding ligand of hPXR-LBD (TO901317) in the presence of BODIPY Fl-vinblastine, GST-hPXR-LBD, and Tb-anti-GST in the presence of the indicated concentrations of DMSO.

FIG. 5 shows representative data for the interaction of BODIPY Fl-vinblastine with GST-hPXR-LBD and Tb-anti-GST in the presence of the indicated hPXR ligands.

FIG. 6 shows representative data for the interaction of BODIPY Fl-vinblastine with GST-hPXR-LBD and Tb-anti-GST. (Panel A) Competitive binding data for a high affinity binding ligand of hPXR-LBD (TO901317), vincristine, and vinblastine with BODIPY Fl-vinblastine in the presence of GST-hPXR-LBD and Tb-anti-GST. (Panel B) Structures of BODIPY FL propionic acid, BODIPY FL hydrazide, and BODIPY FL EDA. (Panel C) Interaction of BODIPY Fl-vinblastine (BDP-VNB), BODIPY FL propionic acid (BDP-Acid), BODIPY FL hydrazide (BODIPY-Hydrazide), and BODIPY FL EDA (BODIPY-EDA) with or without (indicated as “No hPXR”) GST-hPXR-LBD in the presence of vehicle control (DMSO) or a high affinity binding ligand of hPXR-LBD (TO901317). The inset graph in Panel C shows an enlargement of the BDP-VNB graph.

FIG. 7 shows representative data for the interaction of BODIPY Fl-vindoline with GST-PXRLBD in the presence of vehicle control (DMSO; representing total binding), a high affinity binding ligand of PXRLBD (TO901317; representing non-specific binding), or subtraction of non-specific binding from total binding. Assay was performed using a buffer solution containing 50 mM Tris, 50 mM KCl, 1 mM CHAPS, 0.1 mg/mL BSA, and 0.05 mM DTT (pH=7.5). The K_{d} value, derived from the specific binding curve, was 256 nM±12 nM.

FIG. 8 shows representative data for the interaction of BODIPY Fl-vinblastine with GST-PXRLBD in the presence of vehicle control (DMSO; representing total binding), a high affinity binding ligand of PXRLBD (TO901317; representing non-specific binding), or subtraction of non-specific binding from total binding. Assay was performed using a buffer solution containing 50 mM Tris, 50 mM KCl, 1 mM CHAPS, 0.1 mg/mL BSA, and 0.05 mM DTT (pH=7.5).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the terms “PXR” and “pregnane X receptor” can be used interchangeably and refer to a nuclear receptor protein encoded by the NR1I2 gene, which is a transcriptional regulator of cytochrome P450 gene CYP3A4. PXR has a human gene map locus given as 3q12-q13.3, 3q13.3, and 3q12-q13.3 by Entrez Gene, Ensembl, and HGNC, respectively. The corresponding rat and mouse genes are given the gene symbol Nrli2, and the respective gene map loci are 11q21 and 16 B3. The gene and protein have variously been referred to in the scientific literature as ONR1, BXR, SXR, PAR2, Orphan nuclear receptor PAR1, pregnane-activated receptor, steroid and xenobiotic receptor, MGC108643, pregnane X receptor (nuclear receptor sub family 1, group I, member 2), nuclear receptor subfamily 1 group I member 2, orphan nuclear receptor PXR, PXR.1, PXR.2, mPXR, and nuclear receptor subfamily 1, group 1, member 2. It can be appreciated that these terms can also be used to refer to PXR. The term PXR is understood to be inclusive of related homologous proteins in other species. The human form can be specifically designated by the term “hPXR.” The PXR protein is characterized by a DNA binding domain and a ligand binding domain (also referred to by the term “LBD”). The PXR protein forms a heterodimer with the 9-cis retinoic acid receptor RXR, and the formation of the heterodimer is required for transcriptional activation of target genes, and the heterodimer binds to the response element of the CYP3A4 promoter. The heterodimer is also believed to bind to the response elements of the ABCB1/MDR1 gene.

The major human, rat, and mouse PXR protein isoforms encoded by the PXR gene (NR1I2) are, respectively, 434, 431, and 431 amino acids. However, several major splice variants have been described at least for human encoding different isoforms, some of which have been described as using non-AUG translation initiation codons. For example, a significant human isoform is the “long isoform”, that is 473 amino acids comprising 39 additional amino acids added to the N-terminus of the major human isoform which is 434 amino acids. The LBD of the major human isoform is from amino acids 141-434, whereas the LBD of the long isoform is from amino acids 180-473.

As used herein, “hPXR LBD” refers to the protein sequence comprising amino acids 141-434 of the major human protein isoform and to

As used herein, the term “FRET” means “fluorescence resonance energy transfer” or “Forster resonance energy transfer,” and refers to the radiation-less transmission of an energy quantum from its site of absorption (the donor) to the site of its utilization (the acceptor) in a molecule, or system of molecules, by resonance interaction between donor and acceptor species, over distances considerably greater than interatomic, without substantial conversion to thermal energy, and without the donor and acceptor coming into kinetic collision. A donor is a moiety that initially absorbs energy (e.g., optical energy or electronic energy). A luminescent metal complex as described herein can comprise two donors: 1) an organic antenna moiety, which absorbs optical energy (e.g., from a photon); and 2) a lanthanide metal ion, which absorbs electronic energy (e.g., transferred from an organic antenna moiety).

As used herein, the term “acceptor” refers to a chemical or biological moiety that accepts energy via resonance energy transfer. In FRET applications, acceptors may re-emit energy transferred from a donor fluorescent or luminescent moiety as fluorescence and are “fluorescent acceptor moieties.” As used herein, such a donor fluorescent or luminescent moiety and an acceptor fluorescent moiety are referred to as a “FRET pair.” Examples of acceptors include 4,4-difluoro-4-bora-3a,4a-diaza-s-indacene analogues (BODIPY) and related fluorophores, coumarins and related fluorophores; xanthenes such as fluoresceins and fluorescein derivatives; fluorescent proteins such as GFP and GFP derivatives; rhodols, rhodamines, and derivatives thereof; resorufins; cyanines; difluoroboradiazaindacenes; and phthalocyanines. Acceptors, including fluorescent acceptor moieties, can also be useful as fluorescent probes in fluorescence polarization assays.

As used herein, the terms “label” or “labeled” refer to the inclusion of a luminescent metal complex or a fluorescent acceptor moiety on a molecule or substance.

The term “comprising,” with respect to a peptide compound, means that a compound may include additional amino acids and/or other chemical moieties at either or both amino- and carboxy-termini of the given sequence. Of course, these additional amino acids or other chemical moieties should not significantly interfere with the activity of the compound. With respect to a composition of the instant invention, the term “comprising” means that a composition may include additional components. These additional components should not significantly interfere with the activity of the composition.

As used herein, the terms “express,” “expressing” and “expression” mean allowing or causing the information in a gene or DNA sequence to become manifest, for example, producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed.” An expression product can be characterized as intracellular, extracellular or secreted.

As used herein, a “polypeptide” refers to a polymer composed of amino acid residues, structural variants, related naturally-occurring structural variants, and synthetic non-naturally occurring analogs thereof linked via peptide bonds. Synthetic polypeptides can be prepared, for example, using an automated polypeptide synthesizer. The term “protein” typically refers to large polypeptides. The term “peptide” typically refers to short polypeptides.

As used herein, a “fragment” of a polypeptide is meant to refer to any portion of a polypeptide or protein smaller than the full-length polypeptide or protein expression product.

As used herein, an “analog” refers to a modified polypeptide substantially similar in structure to the parent polypeptide. The modified polypeptide may have a similar or altered biological activity, or varying degrees of activity, compared to either the entire parent molecule, or to a fragment thereof. For example, the modified polypeptide may have similar or altered (increased or decreased) binding affinity for a ligand or receptor of the parent polypeptide. Analogs differ in the composition of their amino acid sequences based on one or more mutations Amino acid sequence analogs of a polypeptide can be substitutional, insertional, addition or deletion analogs. Deletion analogs, including fragments of a polypeptide, lack one or more residues of the native protein which are not essential for function or immunogenic activity. Insertional analogs involve the addition of, e.g., amino acid(s) at a non-terminal point in the polypeptide. This analog may include insertion of an immunoreactive epitope or simply a single residue. Addition analogs, including fragments of a polypeptide, include the addition of one or more amino acids at either of both termini of a protein and include, for example, fusion proteins. Substitutions can be conservative or non-conservative based on the physico-chemical or functional relatedness of the amino acid that is being replaced and the amino acid replacing it.

As used herein, a “conservative substitution” of an amino acid is a substitution of one amino acid with another amino acid that has similar physical and chemical properties, e.g. in terms of size, volume, charge, hydrophobicity, hydrophilicity, and the like. Amino acids may be grouped by similarities, e.g. properties like hydrophobic, hydrophilic, acidic, basic, polar, apolar, aromatic, small aliphatic, large aliphatic, etc. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine). The conservative nature of a substitution may depend on the location of the amino acid within a polypeptide sequence.

As used herein, the term “variant” refers to a polypeptide, protein or analog thereof that is modified to comprise additional chemical moieties not normally a part of the molecule. Such moieties may modulate the molecule's solubility, absorption, biological half-life, etc. The moieties may alternatively decrease the toxicity of the molecule and eliminate or attenuate any undesirable side effect of the molecule, etc. Moieties capable of mediating such effects are disclosed in Remington's Pharmaceutical Sciences (1980). Procedure for coupling such moieties to a molecule are well known in the art.

As used herein, the terms “recombinant polynucleotide” or “recombinant nucleic acid” refers to a polynucleotide having sequences that are not naturally joined together. For example, a nucleic acid coding for a polypeptide may be joined with a heterologous regulatory control sequence or other non-coding sequence (e.g., promoter, operator, origin of replication, ribosome binding site, etc.). Two or more polynucleotides joined in such a manner may be included together in a vector, and the vector can be used to transform a suitable host cell. A host cell that comprises the recombinant polynucleotide is referred to as a “recombinant host cell.” Alternatively, a host cell in which a polynucleotide is naturally present may be modified by addition of a heterologous regulatory control sequence that controls expression of the host cell's natural occurring polynucleotide. Such a host cell is also referred to as a “recombinant host cell.” The expression product produced by a recombinant host cell is referred to as a “recombinant polypeptide.”

As used herein, the terms “biologically active derivative” or “biologically active variant” includes any derivative or variant of a molecule having substantially the same functional and/or biological properties of said molecule, such as binding properties, and/or the same structural basis, such as a peptidic backbone or a basic polymeric unit.

As used herein, a “tag” is an amino acid sequence fused to a heterologous protein that facilitates the detection or isolation of the heterologous protein. Tags contemplated for use with the compositions and methods described herein include, but are not limited to epitope tags, affinity tags and fluorescent proteins. An epitope tag is typically a short amino acid sequence that can be detected using antibodies that specifically recognize the tag. An affinity tag is a polypeptide sequence that specifically binds a substrate (for example, a histidine tag has affinity for nickel). Fluorescent proteins include, for example, GFP. Although tags are often grouped into the aforementioned categories, one of skill in the art will recognize that some tags can be members of more than one group. For example, specific antibodies are available for some types of affinity tags (e.g., a histidine tag), therefore these types of tags can be considered both affinity and epitope tags. In some embodiments, the nucleic acid modules disclosed herein encode an epitope tag, such as T7, FLAG, hemagglutinin (HA) VSV-G, V5 or c-myc. Antibodies to these and other epitope tags are commercially available for a variety of sources. In some embodiments, the tag is an affinity tag, such as a histidine tag (e.g., His6), MBP, CBP or GST. In some embodiments, the tag is a fluorescent protein, such as GFP or enhanced GFP.

As used herein, the term “tagging” refers to the process of recombinantly attaching a tag to a protein of interest, such as to facilitate detection or isolation of the protein.

Abbreviations used herein are as follows: “TR-FRET” refers to time-resolved fluorescence resonance energy transfer; “min” refers to minute or minutes; and “NMTB” refers to N-methyl-N-[4-[2,2,2-trifluoro-1-hydroxy-1-(trifluoromethyl)ethyl]phenyl]benzenesulfonamide.

As used herein, BFLV refers to BODIPY FL vinblastine, a compound having a structure represented by a formula:

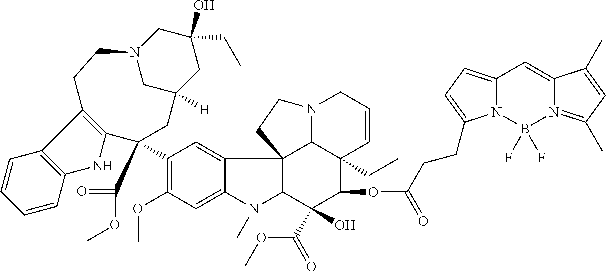

BFLV can also be referred to as BODIPY FL vinblastine or as 7-(3-(((3aR,4R,5S,10bR)-3a-ethyl-9-((3S,5S,7S,9S)-5-ethyl-5-hydroxy-9-(methoxycarbonyl)-2,4,5,6,7,8,9,10-octahydro-1H-3,7-methano[1]azacycloundecino[5,4-b]indol-9-yl)-5-hydroxy-8-methoxy-5-(methoxycarbonyl)-6-methyl-3a,3a¹,4,5,5a,6,11,12-octahydro-1H-indolizino[8,1-cd]carbazol-4-yl)oxy)-3-oxopropyl)-5,5-difluoro-1,3-dimethyl-5H-dipyrrolo[1,2-c:2′,1′-f][1,3,2]diazaborinin-4-ium-5-uide.

As used herein, BODIPY FL propionic acid refers a compound having a structure represented by a formula:

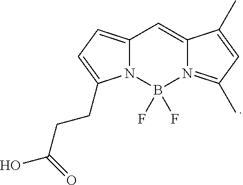

BODIPY FL propionic acid can also be referred to as 7-(2-carboxyethyl)-5,5-difluoro-1,3-dimethyl-5H-dipyrrolo[1,2-c:2′,1′-f][1,3,2]diazaborinin-4-ium-5-uide.

As used herein, BODIPY FL hydrazide refers to a compound having a structure represented by a formula:

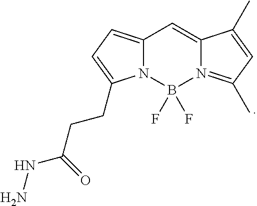

BODIPY FL hydrazide can also be referred to as 5,5-difluoro-7-(3-hydrazinyl-3-oxopropyl)-1,3-dimethyl-5H-dipyrrolo[1,2-c:2′,1′-f][1,3,2]diazaborinin-4-ium-5-uide.

As used herein, BODIPY FL EDA refers to a compound having a structure represented by a formula:

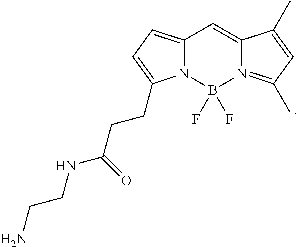

BODIPY FL EDA can also be referred to as 7-(3-((2-aminoethyl)amino)-3-oxopropyl)-5,5-difluoro-1,3-dimethyl-5H-dipyrrolo[1,2-c:2′,1′-f][1,3,2]diazaborinin-4-ium-5-uide.

As used herein, TO901317 refers a compound having a structure represented by a formula:

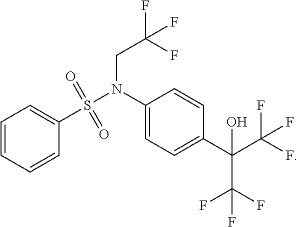 [SMILES: O=S(=O)(c1ccccc1)N(CC(F)(F)F)c1ccc(C(O)(C(F)(F)F)C(F)(F)F)cc1]

TO901317 can also be referred to as N-(2,2,2-trifluoro-ethyl)-N-[4-(2,2,2-tri-fluoro-1-hydroxy-1-trifluoromethyl-ethyl)-phenyl]-benzenesulfonamide. The compound has been described as a potent PXR ligand (e.g. see Mitro, N. et al., FEBS Lett. (2007) 581(9):1721-6).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure

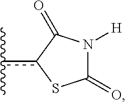

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5,6,7,8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

In some aspects, a structure of a compound can be represented by a formula:

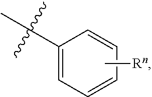

which is understood to be equivalent to a formula:

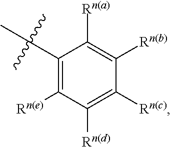

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compositions

In one aspect, the invention relates to compositions useful as fluorescence assay probes. More specifically, in one aspect, the present invention relates to compositions comprising a polypeptide comprising a pregnane X receptor polypeptide, or a ligand binding fragment polypeptide thereof; and a molecule comprising a BODIPY residue and a vinca alkaloid residue.

In one aspect, the disclosed compositions exhibit the ability to assay ligand binding interactions with PXR. In another aspect, the disclosed compositions exhibit the ability to fluoresce to assay the ligand binding interactions with PXR.

In one aspect, the compositions of the invention are useful in the treatment and/or prevention of drug-drug interactions, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

1. Composition Ingredients

In one aspect, the invention relates to a composition comprising a polypeptide comprising a pregnane X receptor polypeptide, or a ligand binding fragment polypeptide thereof; and a molecule comprising a BODIPY residue and a vinca alkaloid residue.

In one aspect, the polypeptide comprises pregnane X receptor polypeptide. In another aspect, the polypeptide comprises a pregnane X receptor ligand binding fragment.

In a further aspect, the pregnane X receptor ligand binding fragment comprises amino acids about 130 to about 473 of human pregnane X receptor, or the homologous amino acids in a non-human pregnane X receptor. In an even further aspect, the pregnane X receptor polypeptide is human pregnane X receptor polypeptide. In a yet further aspect, the pregnane X receptor polypeptide further comprises a heterologous polypeptide.

In another aspect, the heterologous polypeptide comprises one or more affinity tags or epitope tags. In a further aspect, the affinity tag is selected from histidine tag, maltose binding protein tag, chitin binding protein tag, and glutathione-S-transferase tag. In an even further aspect, the epitope tag is selected from T7 tag, FLAG tag, HA tag, VSV-G tag, V5 tag, and c-myc tag.

In a further aspect, the molecule comprising a BODIPY residue and a vinca alkaloid residue is selected from:

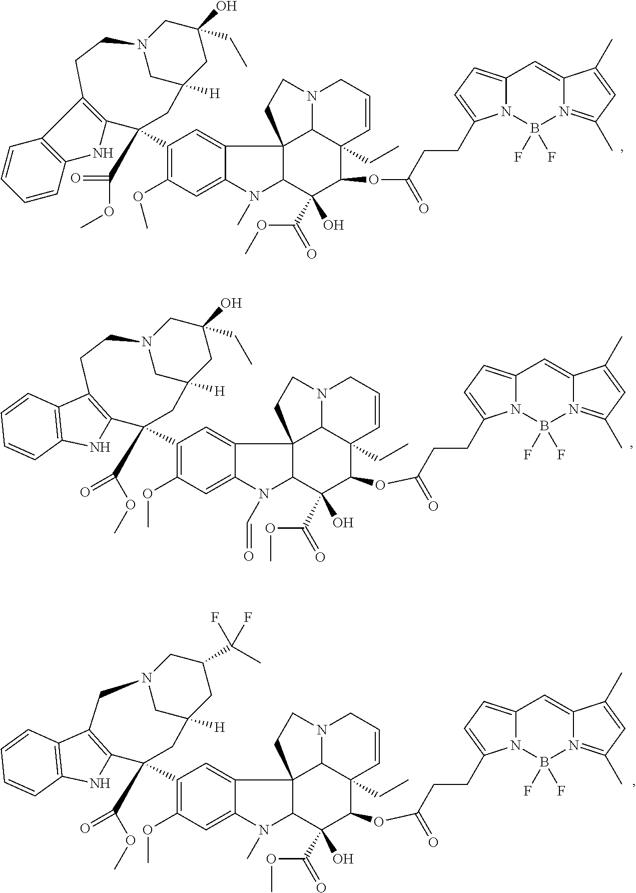

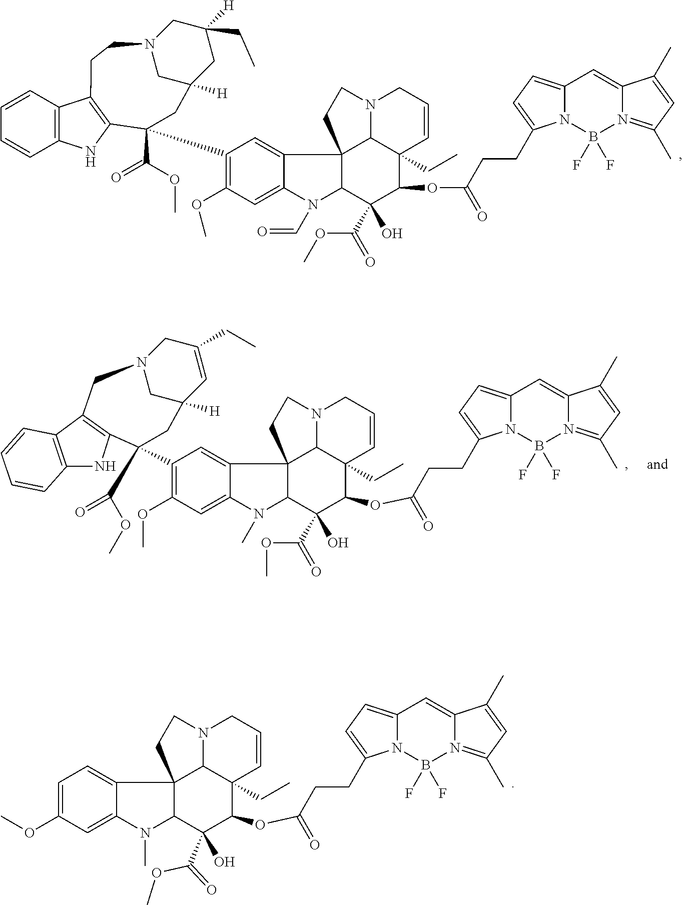

In a further aspect, the molecule comprising a BODIPY residue and a vinca alkaloid residue is:

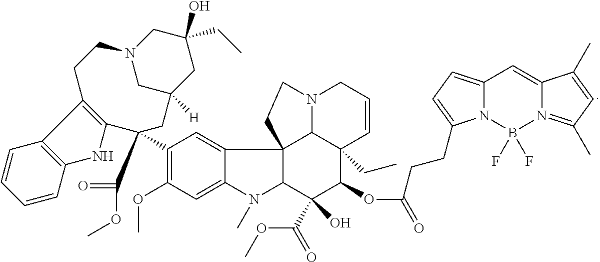

In one aspect, the composition comprises a chimeric polypeptide comprising a pregnane X receptor polypeptide, or a ligand binding fragment polypeptide thereof, and a heterologous polypeptide; a molecule comprising a BODIPY residue and a vinca alkaloid residue; and a molecule comprising chelated terbium and one or more moieties capable of binding the heterologous polypeptide.

In another aspect, the chimeric polypeptide comprises pregnane X receptor polypeptide. In a further aspect, the chimeric polypeptide comprises a pregnane X receptor ligand binding fragment.

In yet another aspect, the pregnane X receptor polypeptide is human pregnane X receptor polypeptide. In another aspect, the pregnane X receptor ligand binding fragment comprises amino acids about 130 to about 473 of human pregnane X receptor, or the homologous amino acids in a non-human pregnane X receptor. In a further aspect, the pregnane X receptor polypeptide is human pregnane X receptor polypeptide.

In one aspect, the heterologous polypeptide comprises one or more affinity tags or epitope tags. In another aspect, the affinity tag is selected from histidine tag, maltose binding protein tag, chitin binding protein tag, and glutathione-S-transferase tag. In a further aspect, the epitope tag is selected from T7 tag, FLAG tag, HA tag, VSV-G tag, V5 tag, and c-myc tag. In an even further aspect,

In a further aspect, the molecule comprising a BODIPY residue and a vinca alkaloid residue is selected from:

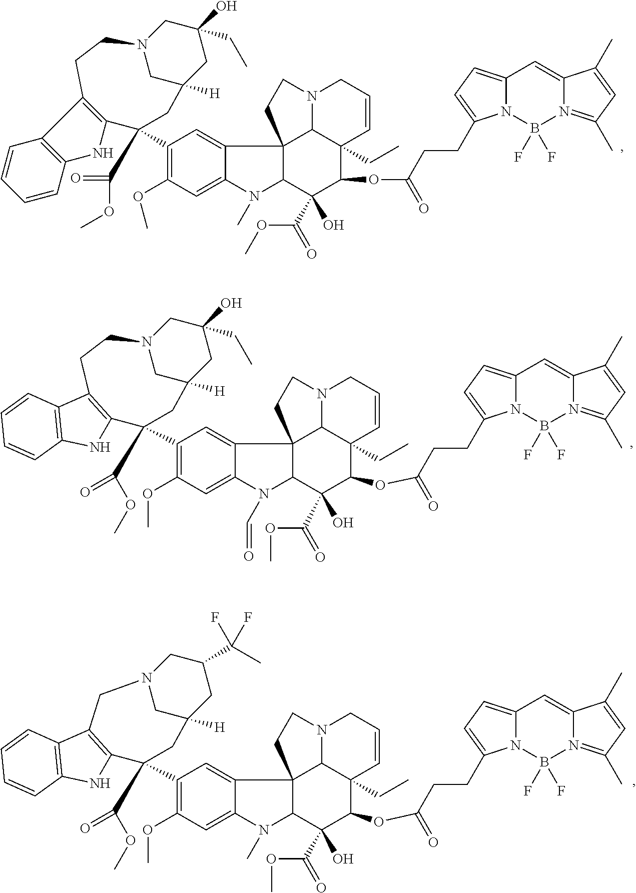

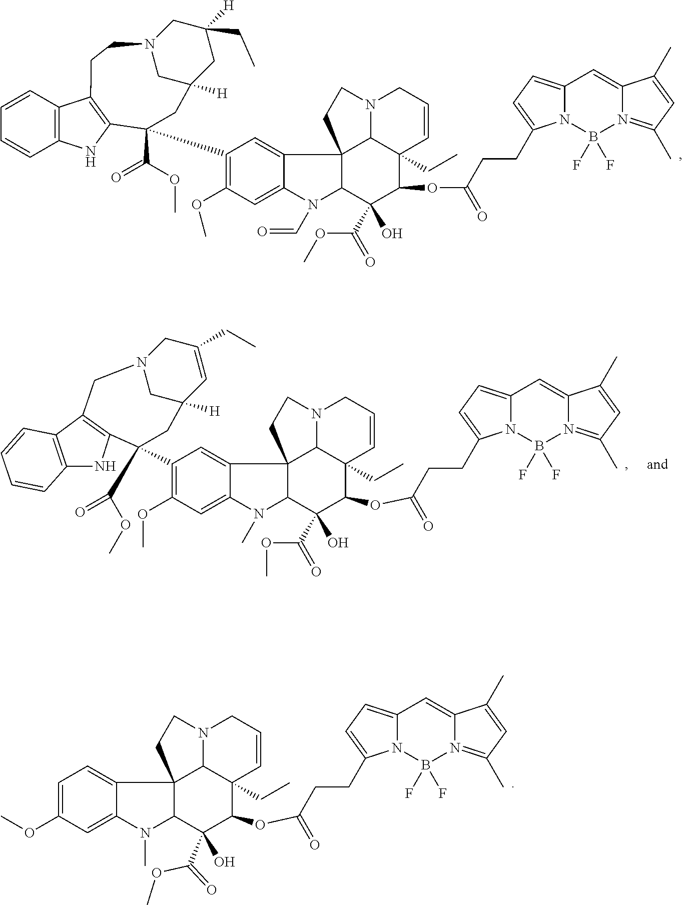

In a further aspect, the molecule comprising a BODIPY residue and a vinca alkaloid residue is:

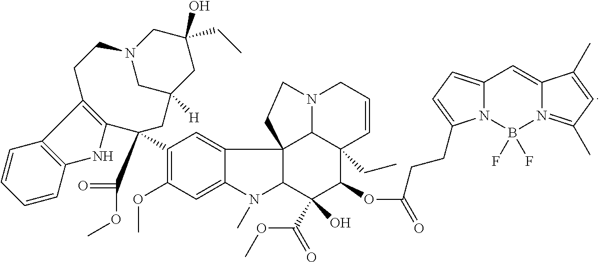

In one aspect, the chelated terbium comprises at least one diethylenetriaminepentaacetic acid residue and Tb⁺³.

In another aspect, the one or more moieties capable of binding the heterologous polypeptide comprise an antibody. In a further aspect, the antibody is an anti-GST antibody. In an even further aspect, the antibody is a monoclonal antibody. In a yet further aspect, the antibody is a polyclonal antibody.

In a further aspect, the composition further comprises a buffer, wherein the buffer comprises 50 mM Tris, 50 mM KCl, 1 mM CHAPS, 0.1 mg/mL BSA, and 0.05 mM DTT. In a still further aspect, the buffer has a pH of about 7.5. In various further aspects, the composition further comprises a buffer, wherein the buffer comprises from about 25 mM to about 150 mM Tris, from about 25 mM to about 200 mM KCl, from about 0.5 mM to about 5 mM CHAPS, from about 0.025 mg/mL to about 0.2 mg/mL BSA, and from about 0.01 mM to about 0.1 mM DTT. In a still further aspect, the buffer has a pH from about 7.2 to about 7.6.

### C. Methods of Making the Compounds

In one aspect, the invention relates to methods of making compositions useful for identifying a test compound that binds to the pregnane X receptor, which can be useful in the treatment and/or prevention of drug-drug interactions.

The molecules in the composition of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, in addition to other standard manipulations known in the literature or to one skilled in the art. The following examples are provided so that the invention might be more fully understood, are illustrative only, and should not be construed as limiting.

In one aspect, the disclosed molecules comprise the products of the synthetic methods described herein. In a further aspect, the disclosed molecules comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

1. Route I

In one aspect, molecules comprising a BODIPY residue and a vinca alkaloid residue of the present invention can be prepared generically as shown below.

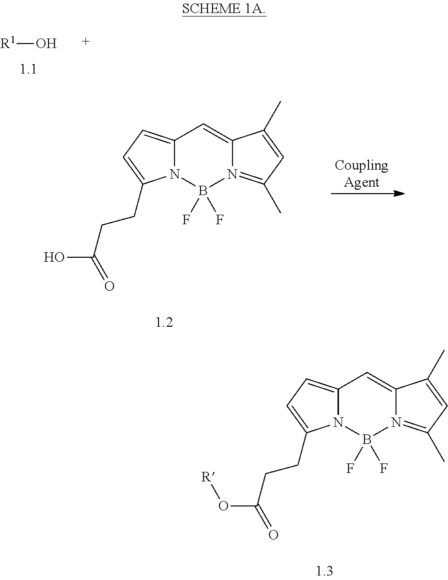

Compounds are represented in generic form, with substituents as noted in compound descriptions elsewhere herein. A more specific example is set forth below.

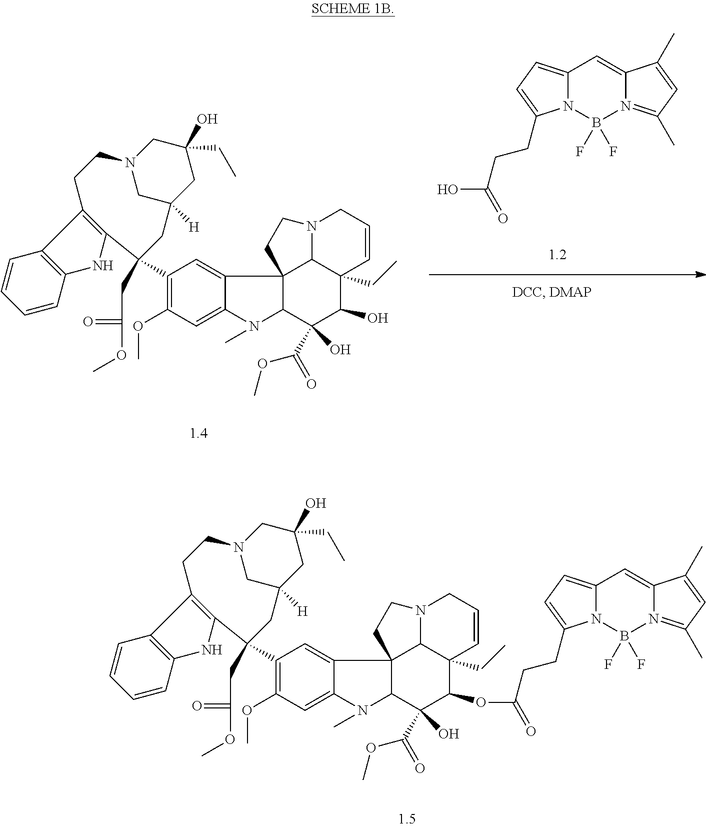

In one aspect, compounds of type 1.3, and similar compounds, can be prepared according to reaction Scheme 1B above. Thus, compounds of type 1.5 can be prepared by a coupling reaction of an appropriate alcohol, e.g., 1.4 as shown above. Appropriate alcohols are commercially available or prepared by methods known to one skilled in the art. The coupling reaction is carried out in the presence of an appropriate carboxylic acid, e.g., BODIPY FL propionic acid (1.2) as shown above, which is commercially available or prepared by methods known to one skilled in the art, and an appropriate coupling reagent, e.g., DCC and DMAP. In another aspect, the reaction can take place in dichloromethane or dimethylformamide (DMF). Further in one aspect, the reaction can be started at 0° C. and warmed to room temperature over several hours. In an even further aspect, the esterification method described in “Simple Method for the Esterification of Carboxylic Acids,” B. Neises, W. Steglich, Angew. Chem. Int. Ed., 1978, 17, 522-524, which is incorporated herein by reference for its teaching of esterification methods. As can be appreciated by one skilled in the art, the above reaction provides an example of a generalized approach wherein compounds similar in structure to the specific reactants above (compounds similar to compounds of type 1.2 and 1.4), can be substituted in the reaction to provide carbazole analogs similar to Formula 1.3.

In a further aspect, the disclosed molecule produced exhibits fluorescence in a composition comprising polypeptide comprising a pregnane X receptor polypeptide, or a ligand binding fragment polypeptide.

It is contemplated that each disclosed methods can further comprise additional steps, manipulations, and/or components. It is also contemplated that any one or more step, manipulation, and/or component can be optionally omitted from the invention. It is understood that a disclosed methods can be used to provide the disclosed compounds. It is also understood that the products of the disclosed methods can be employed in the disclosed methods of using.

### D. Methods of Using the Compounds and Compositions

In one aspect, the invention relates to a method of identifying a test compound that binds to pregnane X receptor, the method comprising the steps of:
a) providing a solution comprising:
   i. a test compound;
   ii. a chimeric polypeptide comprising a pregnane X receptor polypeptide, or a ligand binding fragment polypeptide thereof, and a heterologous polypeptide;
   iii. a fluorescent donor molecule comprising chelated terbium and one or more moieties capable of binding the heterologous polypeptide; and
   iv. a fluorescent acceptor molecule comprising a BODIPY residue and a vinca alkaloid residue;
b) illuminating the solution, thereby causing fluorescence in the fluorescent donor molecule; and
c) measuring fluorescence emission from the fluorescent donor molecule and the fluorescent acceptor molecule.

In one aspect, the illuminating is from about 320 nm to about 360 nm. In another aspect, the illuminating is from about 330 nm to about 350 nm. In a further aspect, the illuminating is at about 340 nm.

In one aspect, the measuring fluorescence emission from the fluorescent donor molecule is from about 470 nm to about 520 nm. In another aspect, the measuring fluorescence emission from the fluorescent donor molecule is from about 480 nm to about 510 nm. In yet another aspect, the measuring fluorescence emission from the fluorescent donor molecule is at about 490 nm.

In one aspect, the measuring fluorescence emission from the fluorescent acceptor molecule is from about 500 nm to about 540 nm. In another aspect, the measuring fluorescence emission from the fluorescent acceptor molecule is from about 510 nm to about 530 nm. In a further aspect, the measuring fluorescence emission from the fluorescent acceptor molecule is at about 520 nm.

In another aspect, the test compound binds to the chimeric polypeptide; and wherein the fluorescence emission ratio of the fluorescent acceptor molecule to the fluorescent donor molecule is less than the fluorescence emission ratio in the absence of test compound. In a further aspect, the chimeric polypeptide comprises pregnane X receptor. In an even further aspect, the chimeric polypeptide comprises a pregnane X receptor ligand binding fragment.

In one aspect, the pregnane X receptor ligand binding fragment comprises amino acids about 130 to about 473 of human pregnane X receptor of the 473 amino acid isoform, amino acids about 141 to about 434 of human pregnane X receptor of the 434 amino acid isoform, or the homologous amino acids in a non-human pregnane X receptor. In another aspect, the pregnane X receptor polypeptide is human pregnane X receptor polypeptide.

In another aspect, the heterologous polypeptide comprises one or more affinity tags or epitope tags. In a further aspect, the affinity tag is selected from histidine tag, maltose binding protein tag, chitin binding protein tag, and glutathione-S-transferase tag. In an even further aspect, the epitope tag is selected from T7 tag, FLAG tag, HA tag, VSV-G tag, V5 tag, and c-myc tag.

In one aspect, the molecule comprising a BODIPY residue and a vinca alkaloid residue is selected from:

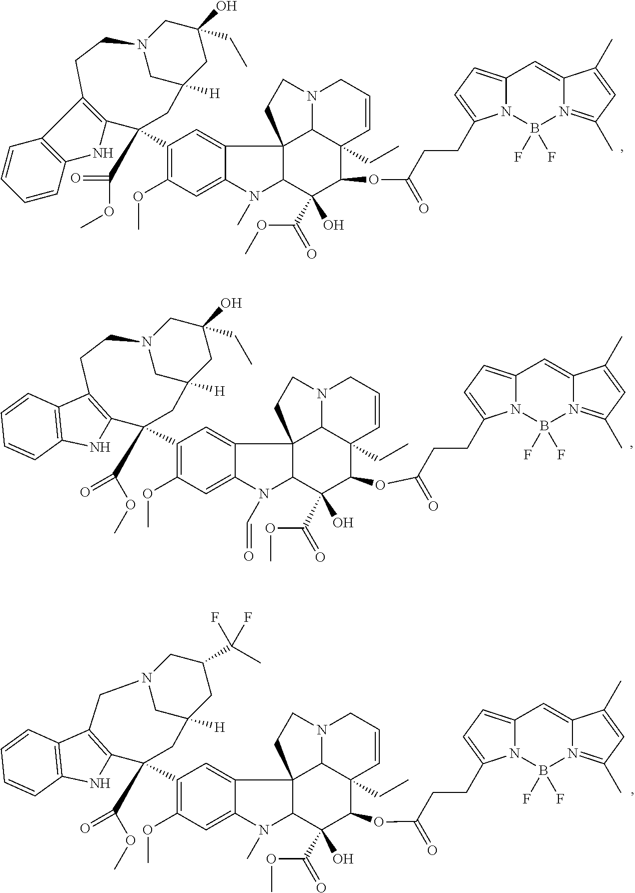

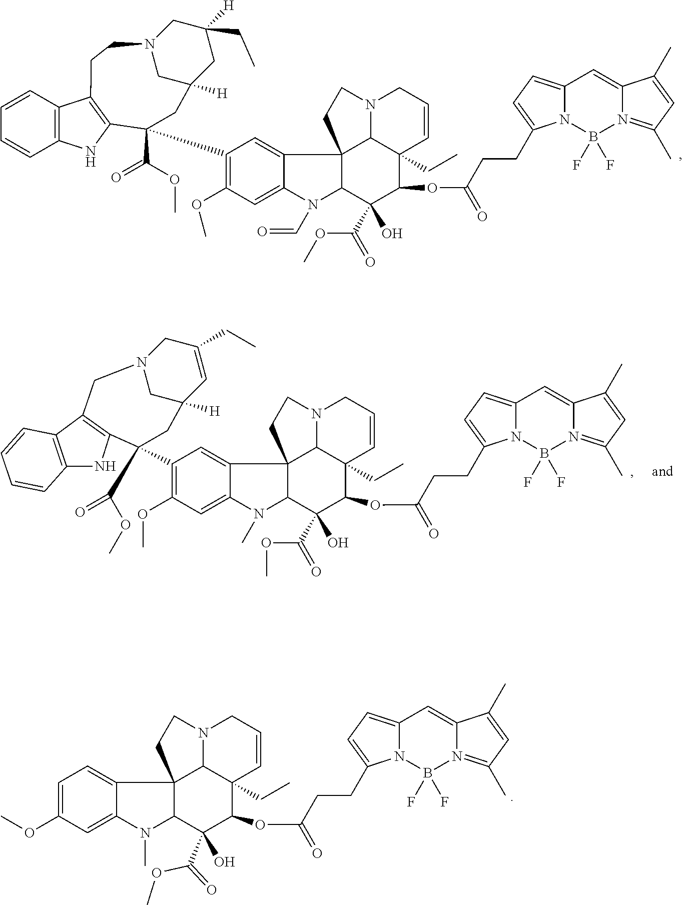

In another aspect, the molecule comprising a BODIPY residue and a vinca alkaloid residue is:

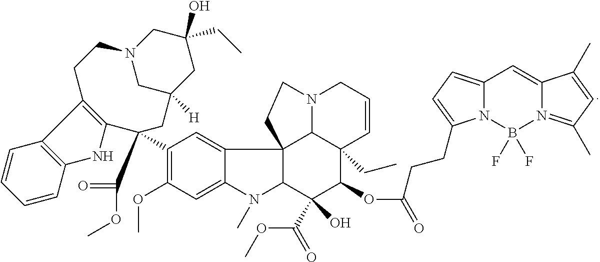

In a further aspect, the chelated terbium comprises at least one diethylenetriaminepentaacetic acid residue and Tb⁺³.

In one aspect, the one or more moieties capable of binding the heterologous polypeptide comprise an antibody. In another aspect, the antibody is an anti-GST antibody. In a further aspect, the antibody is a monoclonal antibody. In an even further aspect, the antibody is a polyclonal antibody.

The disclosed compositions can be used as single agents or in combination with one or more other drugs in the treatment, prevention, control, amelioration or reduction of risk of the aforementioned diseases, disorders and conditions for which compounds of formula I or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

1. Use of Compositions

In one aspect, the invention relates to the use of a disclosed composition or a product of a disclosed method. In a further aspect, a use relates to the manufacture of an assay for the treatment of a disorder associated with drug-drug interaction in a mammal. In a further aspect, the disorder is a binding of PXR disorder caused by the drug-drug interaction. In a further aspect, a use relates to treatment of a binding of PXR disorder associated with drug-drug interaction dysfunction in a mammal.

In a further aspect, a use relates to assaying the fluorescence of the binding activity in a mammal. In a further aspect, a use relates to assaying the fluorescence of binding PXR activity in a mammal. In a further aspect, a use relates to assaying PXR binding activity in a mammal.

In one aspect, the invention relates to the use of a disclosed composition or a disclosed product in the manufacture of a medicament for the treatment of a disorder associated with PXR binding dysfunction in a mammal.

2. Kits

In one aspect, the invention relates to a kit comprising a disclosed composition or a product of a disclosed method and one or more of at least one agent known to assay PXR binding activity; at least one assay to be able to prevent PXR binding activity; at least one agent known to treat a disease of uncontrolled cellular proliferation; or instructions for assaying a disorder associated with PXR binding dysfunction. In a further aspect, the kit further comprises a buffer, wherein the buffer comprises 50 mM Tris, 50 mM KCl, 1 mM CHAPS, 0.1 mg/mL BSA, and 0.05 DTT (pH=7.5). In a further aspect, the at least one composition or the at least one product and the at least one agent are co-formulated. In a further aspect, the at least one composition or the at least one product and the at least one agent are co-packaged.

In one aspect, the invention relates to a kit for measuring the binding activity of a test compound to a pregnane X receptor polypeptide, comprising:
d) a chimeric polypeptide comprising a pregnane X receptor polypeptide, or a ligand binding fragment polypeptide thereof, and a heterologous polypeptide;
e) a fluorescent donor molecule comprising chelated terbium and one or more moieties capable of binding the heterologous polypeptide; and
f) a fluorescent acceptor molecule comprising a BODIPY residue and a vinca alkaloid residue;

and optionally, one or more of:
g) a compound known to bind pregnane X receptor;
h) instructions for measuring a fluorescence emission ratio of the fluorescent acceptor molecule to the fluorescent donor molecule in the presence of a test compound; and
i) instructions for determining the Kᵢ or IC₅₀ of a test compound from a fluorescence emission ratio of the fluorescent acceptor molecule to the fluorescent donor molecule in the presence of a test compound.

In one aspect, the fluorescent acceptor molecule comprises pregnane X receptor polypeptide. In another aspect, the fluorescent acceptor molecule comprises a pregnane X receptor ligand binding fragment.

In a further aspect, the pregnane X receptor ligand binding fragment comprises amino acids about 130 to about 473 of human pregnane X receptor, or the homologous amino acids in a non-human pregnane X receptor. In another aspect, the pregnane X receptor polypeptide is human pregnane X receptor polypeptide. In a further aspect, the pregnane X receptor polypeptide further comprises a heterologous polypeptide.

In one aspect, the heterologous polypeptide comprises one or more affinity tags or epitope tags. In another aspect, the affinity tag is selected from histidine tag, maltose binding protein tag, chitin binding protein tag, and glutathione-S-transferase tag. In a further aspect, the epitope tag is selected from T7 tag, FLAG tag, HA tag, VSV-G tag, V5 tag, and c-myc tag.

In another aspect, the molecule comprising a BODIPY residue and a vinca alkaloid residue is selected from:

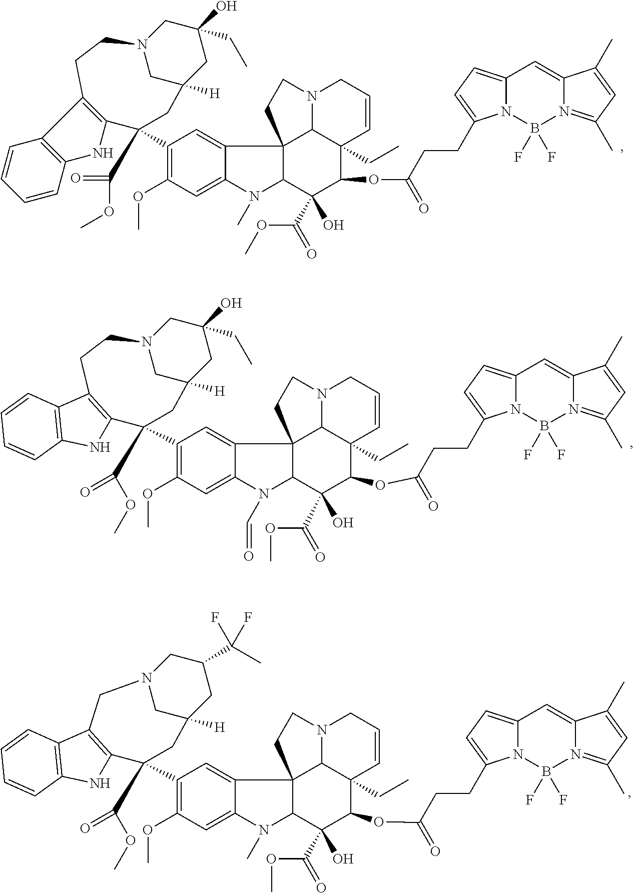

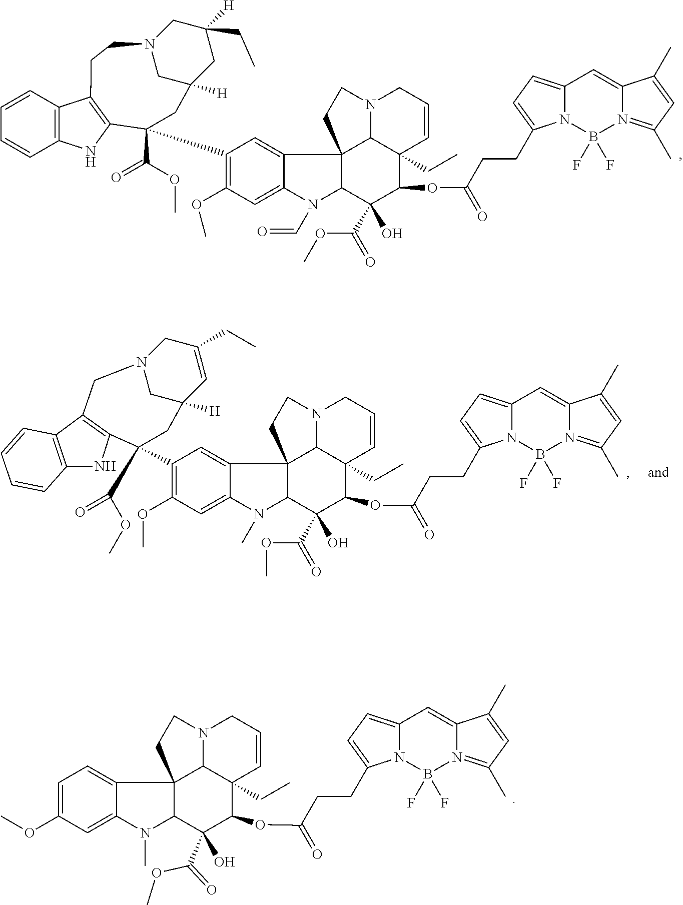

In one aspect, the molecule comprising a BODIPY residue and a vinca alkaloid residue is:

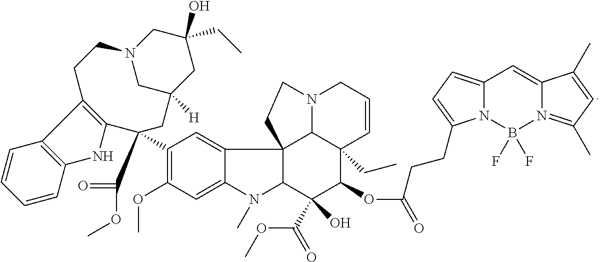

In one aspect, the chimeric polypeptide comprises a pregnane X receptor polypeptide. In another aspect, the chimeric polypeptide comprises a pregnane X receptor ligand binding fragment. In a further aspect, the pregnane X receptor ligand binding fragment comprises amino acids about 130 to about 473 of human pregnane X receptor, or the homologous amino acids in a non-human pregnane X receptor. In an even further aspect, the pregnane X receptor polypeptide is human pregnane X receptor polypeptide.

In one aspect, the heterologous polypeptide comprises one or more affinity tags or epitope tags. In another aspect, the affinity tag is selected from histidine tag, maltose binding protein tag, chitin binding protein tag, and glutathione-S-transferase tag. In a further aspect, the epitope tag is selected from T7 tag, FLAG tag, HA tag, VSV-G tag, V5 tag, and c-myc tag.

In another aspect, the fluorescent acceptor molecule comprising a BODIPY residue and a vinca alkaloid residue is selected from:

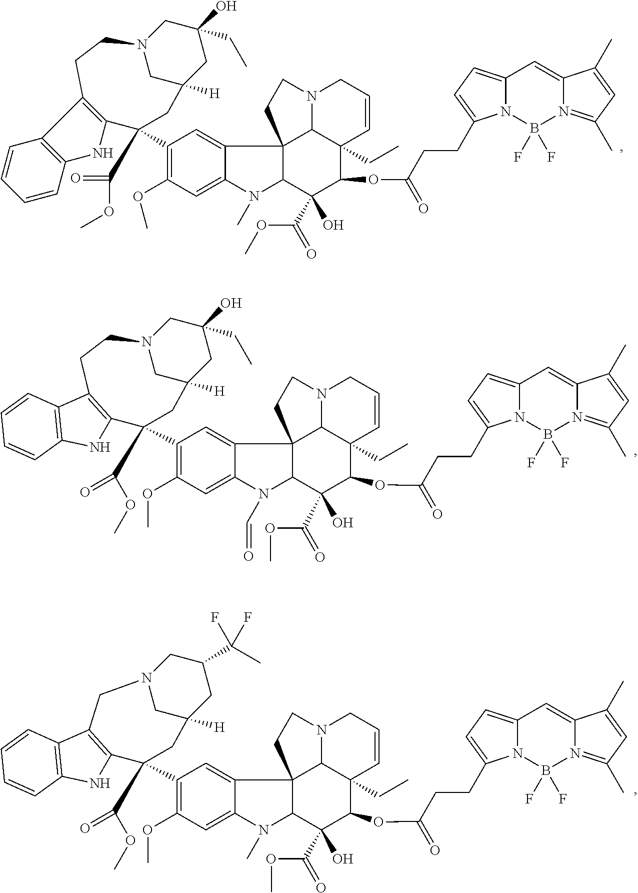

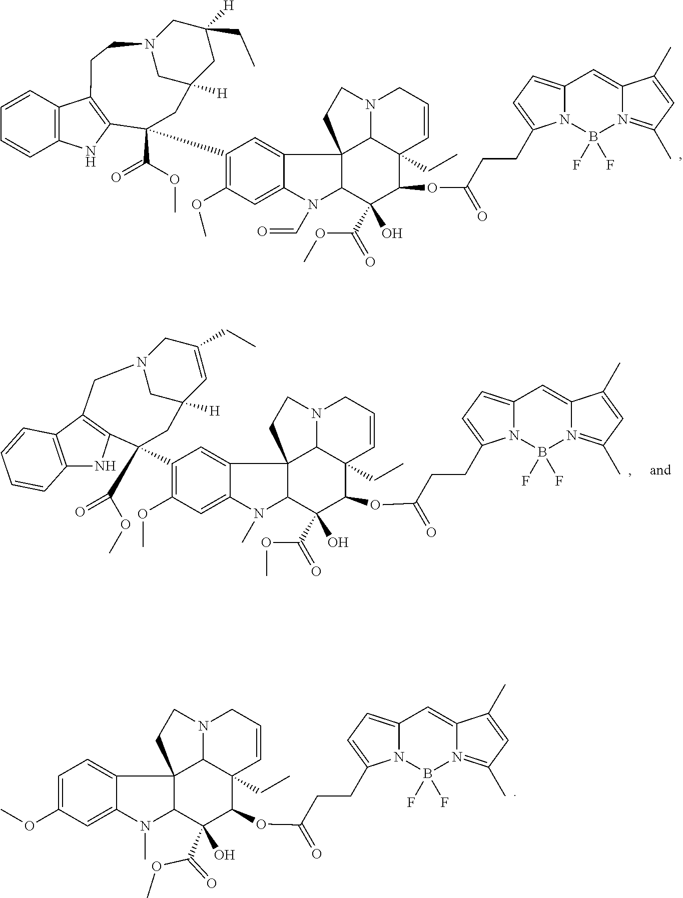

In a further aspect, the fluorescent acceptor molecule comprising a BODIPY residue and a vinca alkaloid residue is:

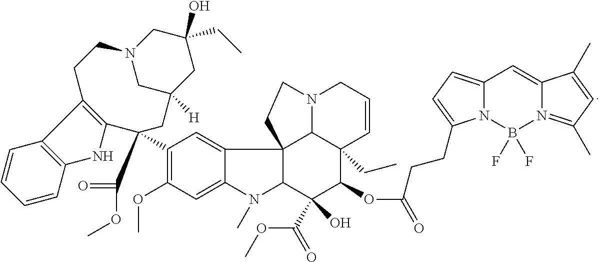

In one aspect, the chelated terbium comprises at least one diethylenetriaminepentaacetic acid residue and Tb+3. In another aspect, the one or more moieties capable of binding the heterologous polypeptide comprise an antibody. In a further aspect, the antibody is an anti-GST antibody. In an even further aspect, the antibody is a monoclonal antibody. In a yet further aspect, the antibody is a polyclonal antibody.

In one aspect, the invention relates to a kit the instructions for measuring a fluorescence emission ratio comprise a method of identifying a test compound that binds to pregnane X receptor, the method comprising the steps of:
a) providing a solution comprising:
   i. a test compound;
   ii. a chimeric polypeptide comprising a pregnane X receptor polypeptide, or a ligand binding fragment polypeptide thereof, and a heterologous polypeptide;
   iii. a fluorescent donor molecule comprising chelated terbium and one or more moieties capable of binding the heterologous polypeptide; and
   iv. a fluorescent acceptor molecule comprising a BODIPY residue and a vinca alkaloid residue;
b) illuminating the solution, thereby causing fluorescence in the fluorescent donor molecule; and
c) measuring fluorescence emission from the fluorescent donor molecule and the fluorescent acceptor molecule.

In one aspect, the fluorescent acceptor molecule comprises pregnane X receptor polypeptide. In another aspect, the fluorescent acceptor molecule comprises a pregnane X receptor ligand binding fragment.

In a further aspect, the pregnane X receptor ligand binding fragment comprises amino acids about 130 to about 473 of human pregnane X receptor, or the homologous amino acids in a non-human pregnane X receptor. In another aspect, the pregnane X receptor polypeptide is human pregnane X receptor polypeptide. In a further aspect, the pregnane X receptor polypeptide further comprises a heterologous polypeptide.

In one aspect, the heterologous polypeptide comprises one or more affinity tags or epitope tags. In another aspect, the affinity tag is selected from histidine tag, maltose binding protein tag, chitin binding protein tag, and glutathione-S-transferase tag. In a further aspect, the epitope tag is selected from T7 tag, FLAG tag, HA tag, VSV-G tag, V5 tag, and c-myc tag.

In another aspect, the molecule comprising a BODIPY residue and a vinca alkaloid residue is selected from:

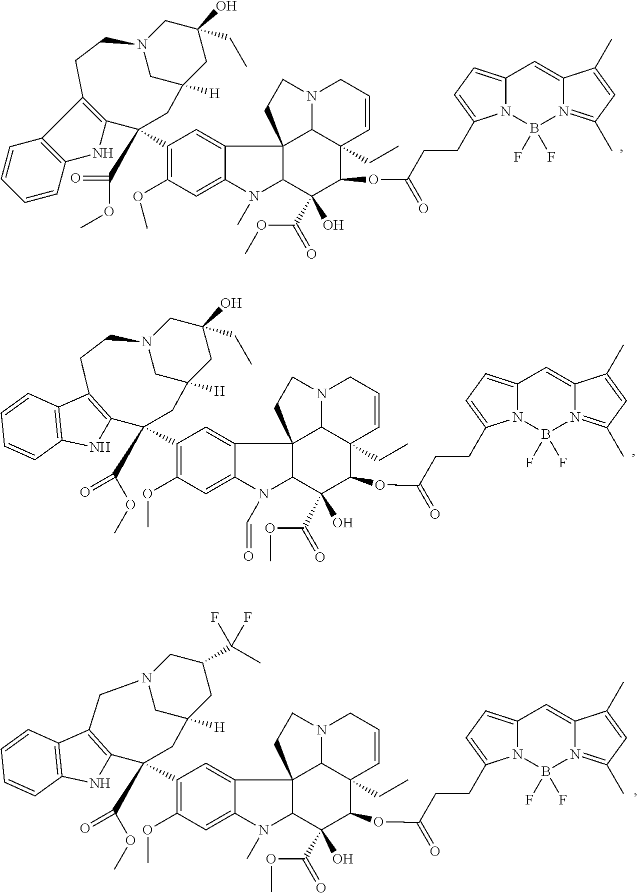

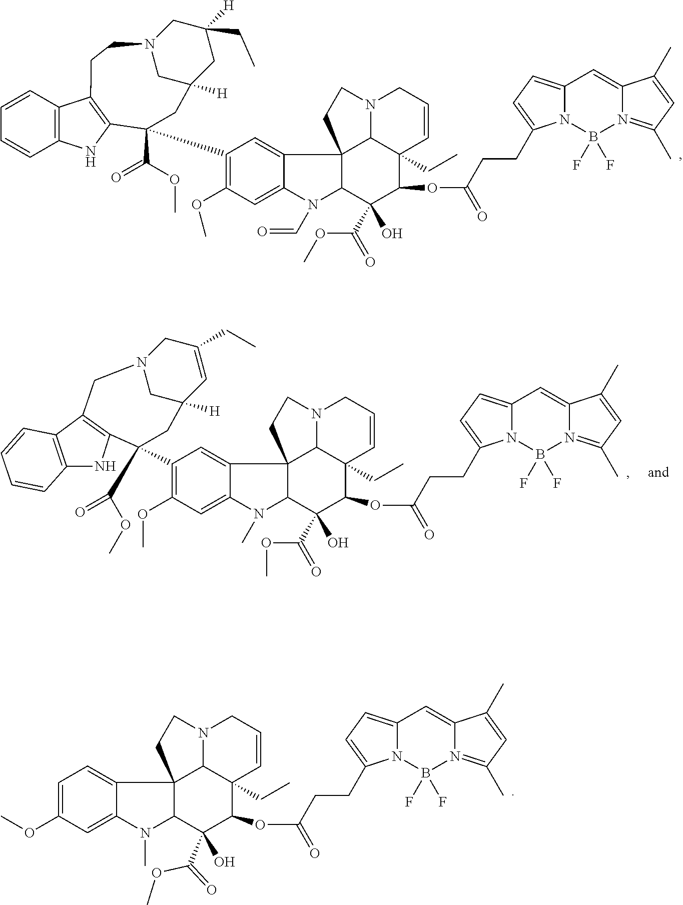

In one aspect, the molecule comprising a BODIPY residue and a vinca alkaloid residue is:

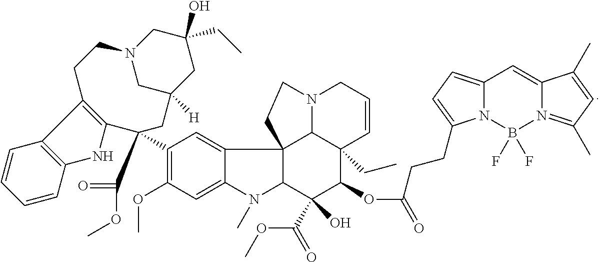

In one aspect, the chimeric polypeptide comprises a pregnane X receptor polypeptide. In another aspect, the chimeric polypeptide comprises a pregnane X receptor ligand binding fragment. In a further aspect, the pregnane X receptor ligand binding fragment comprises amino acids about 130 to about 473 of human pregnane X receptor, or the homologous amino acids in a non-human pregnane X receptor. In an even further aspect, the pregnane X receptor polypeptide is human pregnane X receptor polypeptide.

In one aspect, the heterologous polypeptide comprises one or more affinity tags or epitope tags. In another aspect, the affinity tag is selected from histidine tag, maltose binding protein tag, chitin binding protein tag, and glutathione-S-transferase tag. In a further aspect, the epitope tag is selected from T7 tag, FLAG tag, HA tag, VSV-G tag, V5 tag, and c-myc tag.

In another aspect, the fluorescent acceptor molecule comprising a BODIPY residue and a vinca alkaloid residue is selected from:

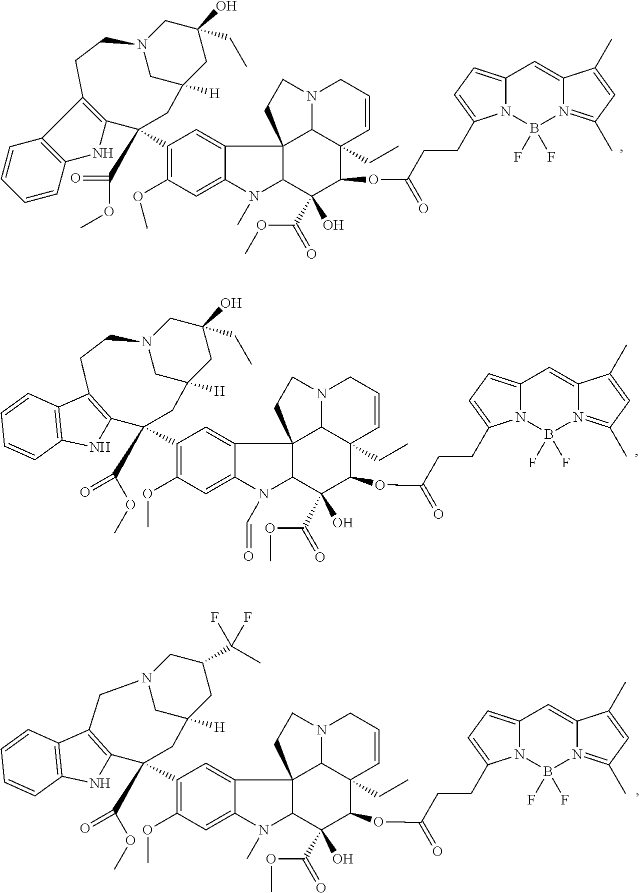

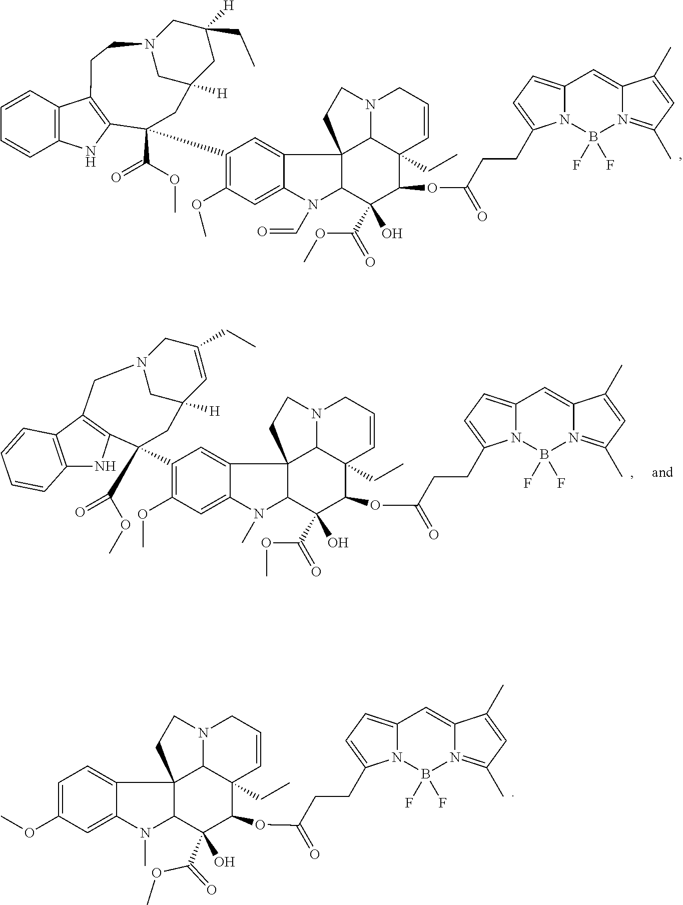

In a further aspect, the fluorescent acceptor molecule comprising a BODIPY residue and a vinca alkaloid residue is:

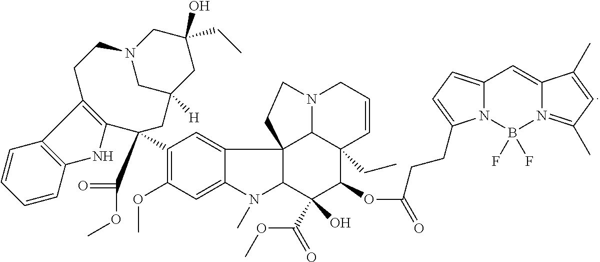

In one aspect, the chelated terbium comprises at least one diethylenetriaminepentaacetic acid residue and Tb+3. In another aspect, the one or more moieties capable of binding the heterologous polypeptide comprise an antibody. In a further aspect, the antibody is an anti-GST antibody. In an even further aspect, the antibody is a monoclonal antibody. In a yet further aspect, the antibody is a polyclonal antibody.

In a further aspect, the kit comprises a disclosed compound or a product of a disclosed method.

In a further aspect, the at least one compound and the at least one agent are co-formulated. In a still further aspect, the at least one compound and the at least one agent are co-packaged.

In various further aspects, the kits can further comprise a suitable buffer for conducting an assay. In a further aspect, the buffer comprises 50 mM Tris, 50 mM KCl, 1 mM CHAPS, 0.1 mg/mL BSA, and 0.05 mM DTT. In a still further aspect, the buffer has a pH of about 7.5. In various further aspects, the composition further comprises a buffer, wherein the buffer comprises from about 25 mM to about 150 mM Tris, from about 25 mM to about 200 mM KCl, from about 0.5 mM to about 5 mM CHAPS, from about 0.025 mg/mL to about 0.2 mg/mL BSA, and from about 0.01 mM to about 0.1 mM DTT. In a still further aspect, the buffer has a pH from about 7.2 to about 7.6.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

3. Non-Medical Uses

Also provided are the uses of the disclosed compositions and products as pharmacological tools in the development and standardization of in vitro and in vivo test systems for the evaluation of the effect of PXR binding related activity in laboratory animals such as cats, dogs, rabbits, monkeys, rats and mice, as part of the search for new therapeutic agents of preventing undesired drug-drug interactions. In a further aspect, the invention relates to the use of a disclosed compound or a disclosed product as pharmacological tools in the development and standardization of in vitro and in vivo test systems for the evaluation of the effect of PXR binding related activity in laboratory animals such as cats, dogs, rabbits, monkeys, rats and mice, as part of the search for new therapeutic agents of preventing ligand binding interactions with PXR.

The disclosed compositions, methods, and articles include at least the following aspects.

Aspect 1: A composition comprising a polypeptide comprising a pregnane X receptor polypeptide, or a ligand binding fragment polypeptide thereof; and a molecule comprising a BODIPY residue and a vinca alkaloid residue.

Aspect 2: The composition of Aspect 1, wherein the polypeptide comprises pregnane X receptor polypeptide.

Aspect 3: The composition of Aspect 1, wherein the polypeptide comprises a pregnane X receptor ligand binding fragment.

Aspect 4: The composition of Aspect 3, wherein pregnane X receptor ligand binding fragment comprises amino acids about 130 to about 473 of human pregnane X receptor, or the homologous amino acids in a non-human pregnane X receptor.

Aspect 5: The composition of any of Aspects 1-2, wherein the pregnane X receptor polypeptide is human pregnane X receptor polypeptide.

Aspect 6: The composition of Aspect 1, wherein the pregnane X receptor polypeptide further comprises a heterologous polypeptide.

Aspect 7: The composition of Aspect 6, wherein the heterologous polypeptide comprises one or more affinity tags or epitope tags.

Aspect 8: The composition of Aspect 7, wherein the affinity tag is selected from histidine tag, maltose binding protein tag, chitin binding protein tag, and glutathione-S-transferase tag.

Aspect 9: The composition of Aspect 7, wherein the epitope tag is selected from T7 tag, FLAG tag, HA tag, VSV-G tag, V5 tag, and c-myc tag.

Aspect 10: The composition of Aspect 1, wherein the molecule comprising a BODIPY residue and a vinca alkaloid residue is selected from:

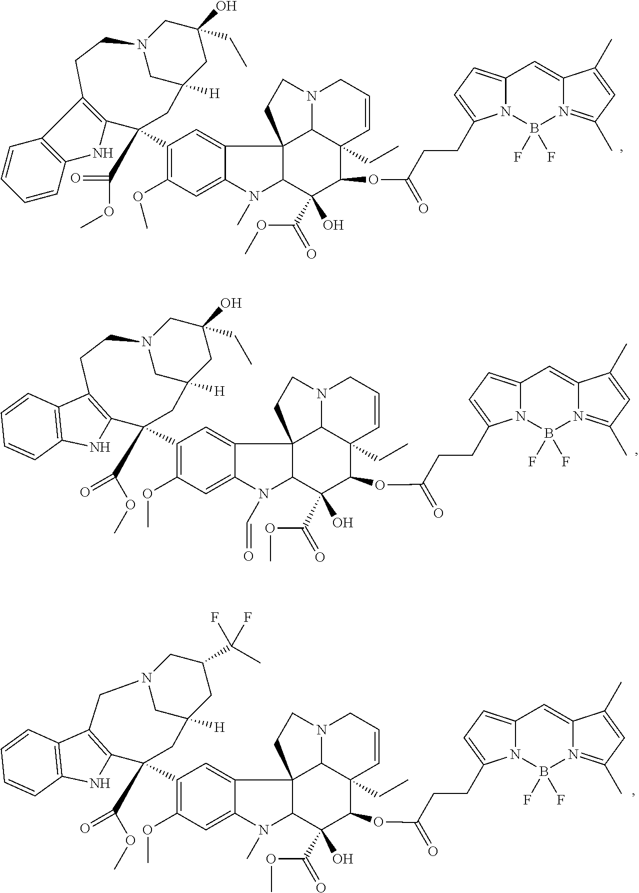

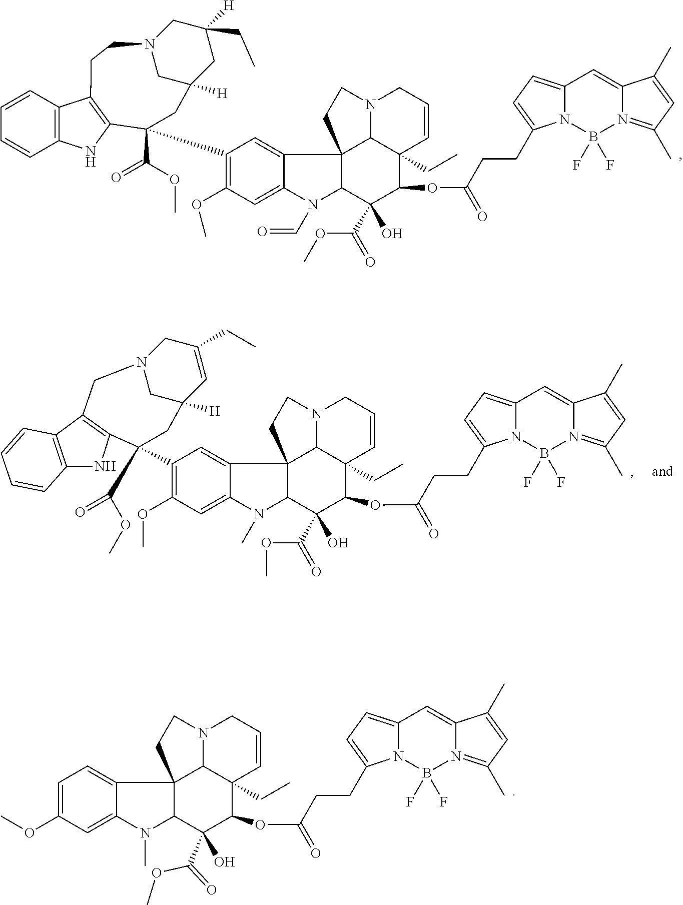

Aspect 11: The composition of Aspect 1 or [00146], wherein the molecule comprising a BODIPY residue and a vinca alkaloid residue is:

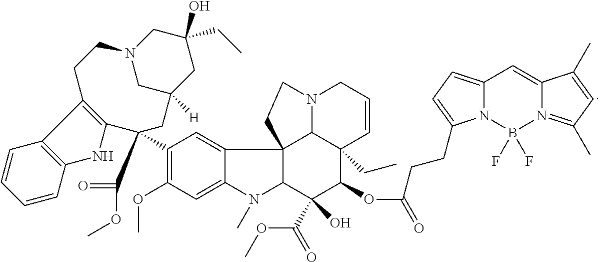

Aspect 12: A composition comprising a chimeric polypeptide comprising a pregnane X receptor polypeptide, or a ligand binding fragment polypeptide thereof, and a heterologous polypeptide; a molecule comprising a BODIPY residue and a vinca alkaloid residue; and a molecule comprising chelated terbium and one or more moieties capable of binding the heterologous polypeptide.

Aspect 13: The composition of Aspect [00148], wherein the polypeptide comprises pregnane X receptor polypeptide.

Aspect 14: The composition of Aspect [00148], wherein the polypeptide comprises a pregnane X receptor ligand binding fragment.

Aspect 15: The composition of Aspect [00150], wherein pregnane X receptor ligand binding fragment comprises amino acids about 130 to about 473 of human pregnane X receptor, or the homologous amino acids in a non-human pregnane X receptor.

Aspect 16: The composition of any of Aspects [00148]-[00151], wherein the pregnane X receptor polypeptide is human pregnane X receptor polypeptide.

Aspect 17: The composition of Aspect [00148], wherein the heterologous polypeptide comprises one or more affinity tags or epitope tags.

Aspect 18: The composition of Aspect [00153], wherein the affinity tag is selected from histidine tag, maltose binding protein tag, chitin binding protein tag, and glutathione-S-transferase tag.

Aspect 19: The composition of Aspect [00153], wherein the epitope tag is selected from T7 tag, FLAG tag, HA tag, VSV-G tag, V5 tag, and c-myc tag.

Aspect 20: The composition of Aspect [00148], wherein the molecule comprising a BODIPY residue and a vinca alkaloid residue is selected from:

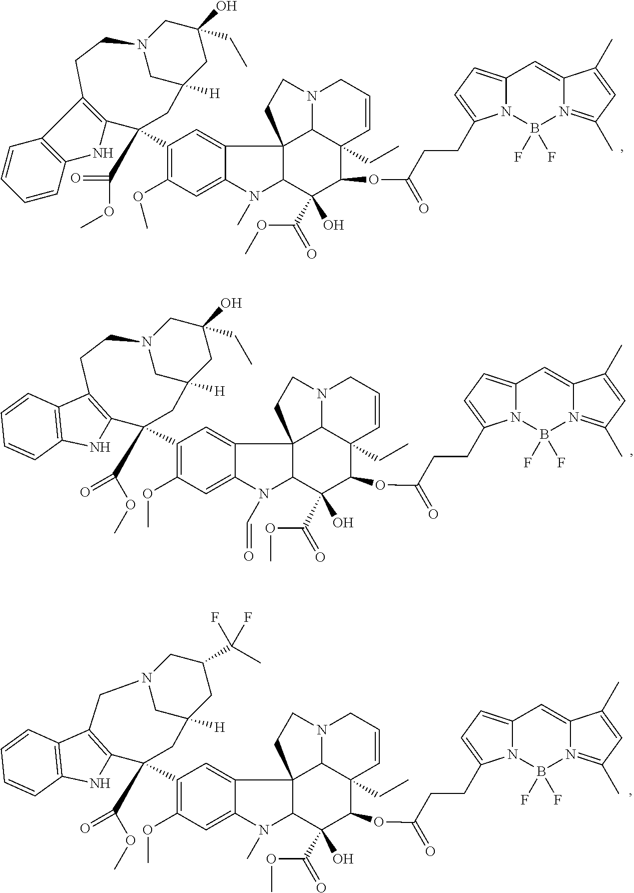

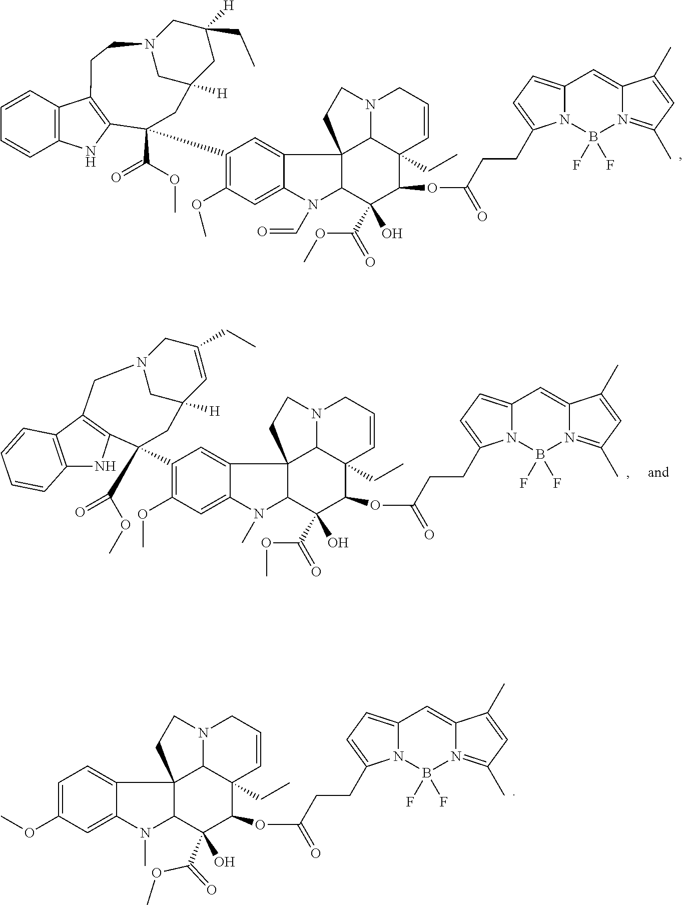

Aspect 21: The composition of Aspect [00148] or [00156], wherein the molecule comprising a BODIPY residue and a vinca alkaloid residue is:

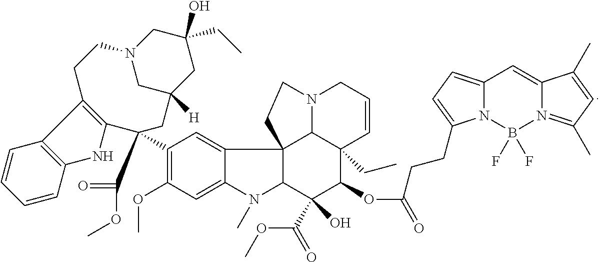

Aspect 22: The composition of Aspect [00148], wherein the chelated terbium comprises at least one diethylenetriaminepentaacetic acid residue and Tb⁺³.

Aspect 23: The composition of Aspect [00148], wherein the one or more moieties capable of binding the heterologous polypeptide comprise an antibody.

Aspect 24: The composition of Aspect [00159], wherein the antibody is an anti-GST antibody.

Aspect 25: The composition of Aspect [00160], wherein the antibody is a monoclonal antibody.

Aspect 26: The composition of Aspect [00160], wherein the antibody is a polyclonal antibody.

Aspect 27: A method of identifying a test compound that binds to pregnane X receptor, the method comprising the steps of: a) providing a solution comprising: i) a test compound; ii) a chimeric polypeptide comprising a pregnane X receptor polypeptide, or a ligand binding fragment polypeptide thereof, and a heterologous polypeptide; iii) a fluorescent donor molecule comprising chelated terbium and one or more moieties capable of binding the heterologous polypeptide; and iv) a fluorescent acceptor molecule comprising a BODIPY residue and a vinca alkaloid residue; b) illuminating the solution, thereby causing fluorescence in the fluorescent donor molecule; and c) measuring fluorescence emission from the fluorescent donor molecule and the fluorescent acceptor molecule.

Aspect 28: The method of Aspect 9, wherein the illuminating is from about 320 nm to about 360 nm.

Aspect 29: The method of Aspect 9, wherein the illuminating is from about 330 nm to about 350 nm.

Aspect 30: The method of Aspect 9, wherein the illuminating is at about 340 nm.

Aspect 31: The method of Aspect 9, wherein the measuring fluorescence emission from the fluorescent donor molecule is from about 470 nm to about 520 nm.

Aspect 32: The method of Aspect 9, wherein the measuring fluorescence emission from the fluorescent donor molecule is from about 480 nm to about 510 nm.

Aspect 33: The method of Aspect 9, wherein the measuring fluorescence emission from the fluorescent donor molecule is at about 490 nm.

Aspect 34: The method of Aspect 9, wherein the measuring fluorescence emission from the fluorescent acceptor molecule is from about 500 nm to about 540 nm.

Aspect 35: The method of Aspect 9, wherein the measuring fluorescence emission from the fluorescent acceptor molecule is from about 510 nm to about 530 nm.

Aspect 36: The method of Aspect 9, wherein the measuring fluorescence emission from the fluorescent acceptor molecule is at about 520 nm.

Aspect 37: The method of Aspect 9, wherein the test compound binds to the chimeric polypeptide; and wherein the fluorescence emission ratio of the fluorescent acceptor molecule to the fluorescent donor molecule is less than the fluorescence emission ratio in the absence of test compound.

Aspect 38: The method of Aspect 9, wherein the polypeptide comprises pregnane X receptor polypeptide.

Aspect 39: The method of Aspect 9, wherein the polypeptide comprises a pregnane X receptor ligand binding fragment.

Aspect 40: The method of Aspect 13, wherein pregnane X receptor ligand binding fragment comprises amino acids about 130 to about 473 of human pregnane X receptor of the 473 amino acid isoform, amino acids about 141 to about 434 of human pregnane X receptor of the 434 amino acid isoform, or the homologous amino acids in a non-human pregnane X receptor.

Aspect 41: The method of any of Aspects 9 or [00174]-14, wherein the pregnane X receptor polypeptide is human pregnane X receptor polypeptide.

Aspect 42: The method of Aspect 9, wherein the heterologous polypeptide comprises one or more affinity tags or epitope tags.

Aspect 43: The method of Aspect 16, wherein the affinity tag is selected from histidine tag, maltose binding protein tag, chitin binding protein tag, and glutathione-S-transferase tag.

Aspect 44: The method of Aspect 16, wherein the epitope tag is selected from T7 tag, FLAG tag, HA tag, VSV-G tag, V5 tag, and c-myc tag.

Aspect 45: The method of Aspect 9, wherein the molecule comprising a BODIPY residue and a vinca alkaloid residue is selected from:

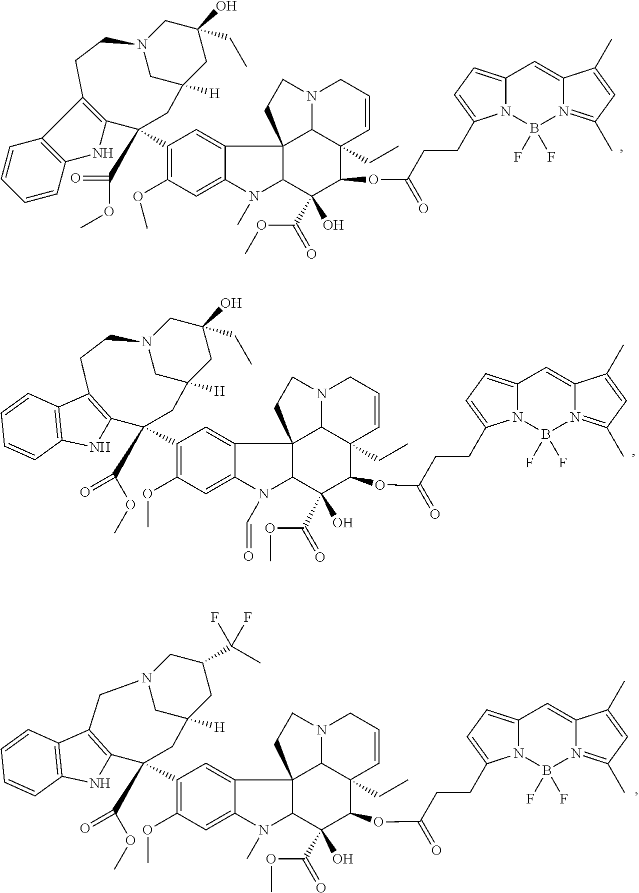

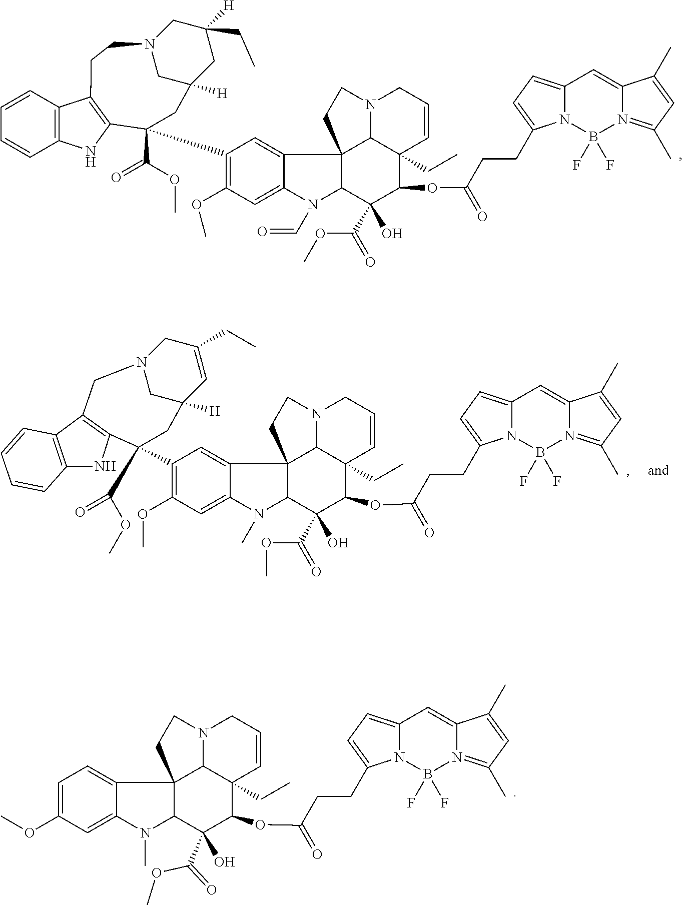

Aspect 46: The method of Aspect 9 or [00181], wherein the molecule comprising a BODIPY residue and a vinca alkaloid residue is:

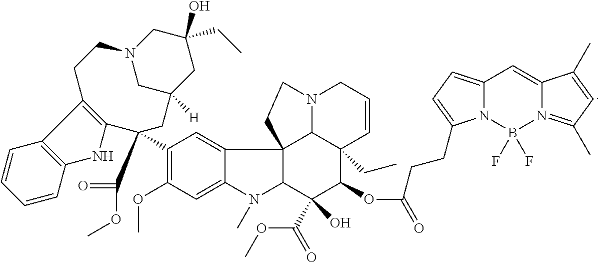

Aspect 47: The method of Aspect 9, wherein the chelated terbium comprises at least one diethylenetriaminepentaacetic acid residue and Tb⁺³.

Aspect 48: The method of Aspect 9, wherein the one or more moieties capable of binding the heterologous polypeptide comprise an antibody.

Aspect 49: The method of Aspect 19, wherein the antibody is an anti-GST antibody.

Aspect 50: The method of Aspect [00185], wherein the antibody is a monoclonal antibody.

Aspect 51: The method of Aspect [00185], wherein the antibody is a polyclonal antibody.

Aspect 52: A kit for measuring the binding activity of a test compound to a pregnane X receptor polypeptide, comprising: a) a chimeric polypeptide comprising a pregnane X receptor polypeptide, or a ligand binding fragment polypeptide thereof, and a heterologous polypeptide; b) a fluorescent donor molecule comprising chelated terbium and one or more moieties capable of binding the heterologous polypeptide; and c) a fluorescent acceptor molecule comprising a BODIPY residue and a vinca alkaloid residue; and optionally, one or more of: d) a compound known to bind pregnane X receptor; e) instructions for measuring a fluorescence emission ratio of the fluorescent acceptor molecule to the fluorescent donor molecule in the presence of a test compound; and f) instructions for determining the Kᵢ, or IC₅₀ of a test compound from a fluorescence emission ratio of the fluorescent acceptor molecule to the fluorescent donor molecule in the presence of a test compound.

### E. Experimental

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Several methods for preparing the compounds of this invention are illustrated in the following Examples. Starting materials and the requisite intermediates are in some cases commercially available, or can be prepared according to literature procedures or as illustrated herein.

The following exemplary compounds of the invention were synthesized. The Examples are provided herein to illustrate the invention, and should not be construed as limiting the invention in any way. The Examples are typically depicted in free base form, according to the IUPAC naming convention. However, some of the Examples were obtained or isolated in salt form.

As indicated, some of the Examples were obtained as racemic mixtures of one or more enantiomers or diastereomers. The compounds may be separated by one skilled in the art to isolate individual enantiomers. Separation can be carried out by the coupling of a racemic mixture of compounds to an enantiomerically pure compound to form a diastereomeric mixture, followed by separation of the individual diastereomers by standard methods, such as fractional crystallization or chromatography. A racemic or diastereomeric mixture of the compounds can also be separated directly by chromatographic methods using chiral stationary phases.

1. Synthesis of BODIPY FL Vindoline

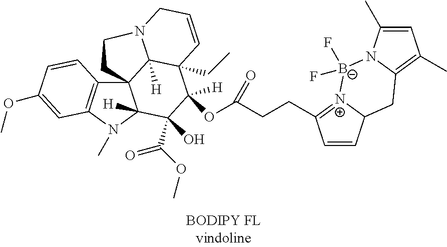

BODIPY FL vindoline was prepared using a N-(3-nimethylaminopropyl)-N′-ethylcarbodiimide hydrochloride (EDAC) and 4-(dimethylamino)pyridine-mediated (DMAP-mediated) coupling reaction (Chrominski, M., et al. (2013) J. Med. Chem. 56, 7260-7277). Briefly, a mixture of deacetyl vindoline (21 mg, 50 μmol), BODIPY FL propionic acid (74 mg, 50 μmol), EDAC (11.5 mg, 60 μmol) and DMAP (7.4 mg, 60 μmol) were dissolved in anhydrous methylene chloride (CH₂Cl₂, 5 mL) and the reaction was stirred at room temperature under nitrogen for 3 hours until mass spectra indicated the starting material deacetyl vindoline was not detectable. The reaction mixture was then diluted with CH₂Cl₂ (50 mL), washed with diluted sodium bicarbonate-brine (pH 8) (20 mL×2), and dried over anhydrous sodium sulfate. The solution was then recovered by filtration and concentrated to afford a brown crude product. The crude product was then purified via preparative HPLC. The fractions containing the product were pooled and evaporated to yield BODIPY FL vindoline (25.3 mg, 74% in yield and 98.4% in purity). ¹H NMR (400 MHz, Chloroform-d plus D₂O) δ 7.06 (s, 1H), 6.93-6.85 (m, 2H), 6.35-6.26 (m, 2H), 6.13-6.03 (m, 2H), 5.82 (ddd, J=1.65, 4.88, 10.27 Hz, 1H), 5.50 (s, 1H), 5.20 (dt, J=2.00, 10.24 Hz, 1H), 3.77 (d, J=4.90 Hz, 6H), 3.73 (s, 1H), 3.55-3.36 (m, 2H), 3.29 (t, J=7.58 Hz, 2H), 2.86-2.72 (m, 3H), 2.66 (s, 3H), 2.63 (d, J=9.07 Hz, 1H), 2.54 (s, 3H), 2.54-2.47 (m, 1H), 2.38-2.28 (m, 2H), 2.24 (s, 3H), 1.66 (dt, J=7.41, 14.43 Hz, 1H), 1.14 (dq, J=7.23, 14.46 Hz, 1H), 0.47 (t, J=7.32 Hz, 3H). ESI-TOF HRMS: m/z 689.3323 (C₃₇H₄₃BF₂N₄O₆+H⁺ requires 689.3322).

2. General Methods

All assays were carried out in 20 μL TR-FRET PXR (SXR) assay buffer with 5 nM GST-hPXR-LBD and 5 nM Tb-anti-GST at room temperature in 384-well black polypropylene plates, and all assays were done in triplicate. The plates were briefly centrifuged after mixing of all assay components. The final DMSO concentration was 1.1% in all assays with the exception of the DMSO tolerance test, in which DMSO concentrations were as specified herein below. The typical assay incubation time was 30 min, with the exception of the longitudinal signal stability assays, in which the incubation time is specified. All assay data were generated by using a PHERAstar plate reader from BMG Labtech (Durham, N.C.) to measure the fluorescent emission ratio (520 nm/490 nm) of each well, using a 340 nm excitation filter, 100 is delay time, and 200 is integration time. Raw data from the plate reader were directly used for analysis. The curve-fitting software GraphPad Prism 5.04 (GraphPad Software, La Jolla, Calif.) was used to generate graphs and curves and to determine K_{d} and IC₅₀ values.

3. Probe K_{D} Determination in the hPXR TR-FRET Assay

Serial dilutions of BFLV (1:2 titration, 15 concentrations of 5000-0.31 nM) were incubated with 5 nM GST-hPXR-LBD and 5 nM Tb-anti-GST plus either DMSO or 50 μM TO901317 for 30 min before TR-FRET signals were collected. The collected data were fit into a one-site total binding equation and the specific equilibrium binding constant (K_{d}) was derived from the specific binding curve, derived as curve_{DMSO}-curve_{TO901317}.

4. Optimization of Probe Concentration for hPXR TR-FRET Binding Assays

100, 250, or 500 nM BFLV was incubated with 5 nM GST-hPXR-LBD and 5 nM Tb-anti-GST plus either DMSO, 10 μM TO901317, or 50 μM TO901317 for 30 min, and then TR-FRET signals were collected.

5. Determination of Signal Stability in the BFLV-Based hPXR TR-FRET Assay

250 nM BFLV was incubated with 5 nM GST-hPXR-LBD and 5 nM Tb-anti-GST plus either DMSO, 50 μM TO901317, or serial dilutions of TO901317 (50 μM to 0.28 nM with 1-to-3 titration for 12 concentration levels), and TR-FRET signals were collected at 0.5, 1, 2, 3, 4, and 5 h. In the Z′-factor signal stability test, 16 data points were included in each high-signal (DMSO, negative control to determine total binding) or low-signal (50 μM TO901317, positive control to determine non-specific binding) group, and the Z′-factor value was calculated by using equation 1 (see Zhang, J. H.; Chung, T. D.; and Oldenburg, K. R. A J. Biomol. Screen. 1999, 4 (2), 67-73.):

$${Z}^{′} = 1 - \frac{3 {σ}^{+} + 3 {σ}^{-}}{{Mean}^{+} - {Mean}^{-}} , \qquad ( 1 )$$

where σ⁺ is the standard deviation of the negative control (DMSO) group; σ⁻ is the standard deviation of the positive control (50 μM TO901317) group; Mean⁺ is the mean of the negative-control (DMSO) group; and Mean is the mean of the positive-control (50 TO901317) group. The data from TO901317 titration were fit into a one-site competition binding equation to derive IC₅₀ values.

6. DMSO Tolerance Test for the BFLV-Based hPXR TR-FRET Assay

250 nM BFLV was incubated with 5 nM GST-hPXR-LBD and 5 nM Tb-anti-GST plus either DMSO, 50 μM TO901317, or serial dilutions of TO901317 (50 μM to 0.28 nM, with 1:3 titration at 12 concentration levels). The final DMSO concentration was 0.2%, 0.5%, 1%, 2%, 5% or 10% in each group. After incubation for 30 minutes, the TR-FRET signals were collected. The data from titrated TO901317 were fit into a one-site competition binding equation to determine the IC₅₀ values.

7. Binding Affinity of a Panel of hPXR Ligands, Vincristine, and Vinblastine with hPXR in the BFLV-Based TR-FRET Assay

Serial dilutions of TO901317, SR12813, clotrimazole, rifampicin, hyperforin, ginkgolide A, ginkgolide B, vincristine, vinblastine (100 μM to 0.56 nM, 1:3 titration at 12 concentration levels), DMSO, or 50 μM TO901317 were incubated with 250 nM BFLV, 5 nM GST-hPXR-LBD, and 5 nM Tb-anti-GST for 30 min, and then TR-FRET signals were collected. Where applicable, the data were fit into a one-site competitive binding equation in a dose-dependent manner to derive IC₅₀ values. The inhibition constant (IQ value was subsequently calculated by using equation 2 (see Cheng, Y. and Prusoff, W. H. Biochem. Pharmacol. 1973, 22 (23), 3099-3108.):

Kᵢ=IC₅₀/(1+[L]/K_{L}) (2),

where IC₅₀ is the concentration of inhibitor that inhibits 50% of binding, [L] is the concentration of BFLV (250 nM), and K_{L} is the K_{d} value of BFLV in the assay (673 nM). The Kᵢ values were used to compare the binding affinity of compounds to GST-hPXR-LBD.

8. Non-Specific Binding of BODIPY Dyes in the hPXR TR-FRET Assay

250 nM BFLV, BODIPY FL propionic acid, BODIPY FL hydrazide, or BODIPY FL EDA was incubated with 5 nM GST-hPXR-LBD and 5 nM Tb-anti-GST plus either DMSO or 50 μM TO901317 for 30 min, and then TR-FRET signals were collected. In a paralleled control group, 250 nM BFLV, BODIPY FL propionic acid, BODIPY FL hydrazide, or BODIPY FL EDA was incubated with only 5 nM GST-hPXR-LBD plus either DMSO or 50 μM TO901317 for 30 min, and then TR-FRET signals were collected.

9. BODIPY FL Vinblastine Binds to the hPXR Ligand-Binding Domain with High Affinity

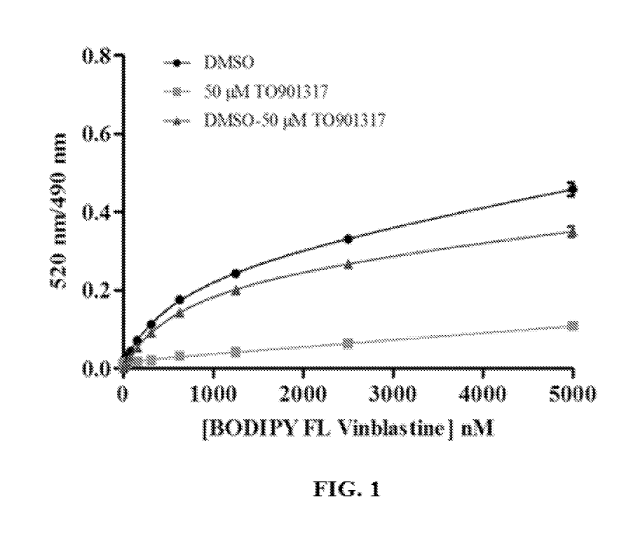

A TR-FRET assay was used to evaluate the ability of disclosed compounds to bind to the ligand-binding domain (LBD) of hPXR. The data show that BODIPY FL vinblastine (BFLV) is a hPXR ligand (FIG. 1). The specific equilibrium binding constant (K_{d}) of BFLV and hPXR-LBD in the TR-FRET assay system was determined by individually plotting the total binding curve (-.- in FIG. 1, in the presence of DMSO vehicle control) and non-specific binding curve (-- in FIG. 1, in the presence of the high-affinity hPXR ligand TO901317) from experimental data, then generating the specific binding curve (-▴- in FIG. 1B) by subtraction of non-specific binding from total binding (DMSO—TO901317). The total binding curve and the non-specific binding curve were derived from titrated BFLV (5000-0.31 nM, with 1:2 dilution, at 15 concentration levels) incubated with 5 nM GST-hPXR-LBD and 5 nM Tb-anti-GST plus either DMSO or 50 μM TO901317, respectively.

TO901317 is a potent hPXR agonist which at 50 μM inhibited all specific binding of BFLV to hPXR LBD at the concentration range tested; any binding activity in the presence of 50 μM TO901317 was therefore considered non-specific. The K_{d} value of BFLV, derived from the specific binding curve, was 673 nM±18 nM, indicating relatively high binding affinity. In the total binding experiments, the TR-FRET signal increased exponentially when the concentration of BFLV increased from nanomolar to low micromolar, and it then increased slowly. In contrast, in the non-specific binding experiments the overall TR-FRET signal was relatively low and increased slowly in a linear manner over the concentration range tested. Because of the low non-specific binding, the specific binding curve was very similar to the total binding curve. These results indicate that BFLV is a high-affinity hPXR fluorescent probe and that it has low non-specific activity in the assay system.

BFLV is not the first high-affinity fluorescent hPXR probe. However, the undisclosed chemical structure of the Fluormone PXR (SXR) Green probe (see LanthaScreen TR-FRET PXR (SXR) Competitive Binding Assay Kit. http://tools.invitrogen.com/content/sfs/manuals/lanthascreen_PXR_man.pdf, (accessed September 2012) makes it less valuable for studying ligand-PXR interactions. In various aspects, the present invention pertains to a hPXR fluorescent probe with a disclosed that is available in pure solid form, allowing the preparation of stock solutions of higher concentration in a preferred solvent. Therefore, BFLV is a highly valuable fluorescent probe for studying regulation of PXR by ligand binding.

10. Determination of the Optimal BFLV Concentration for the hPXR TR-FRET Assay

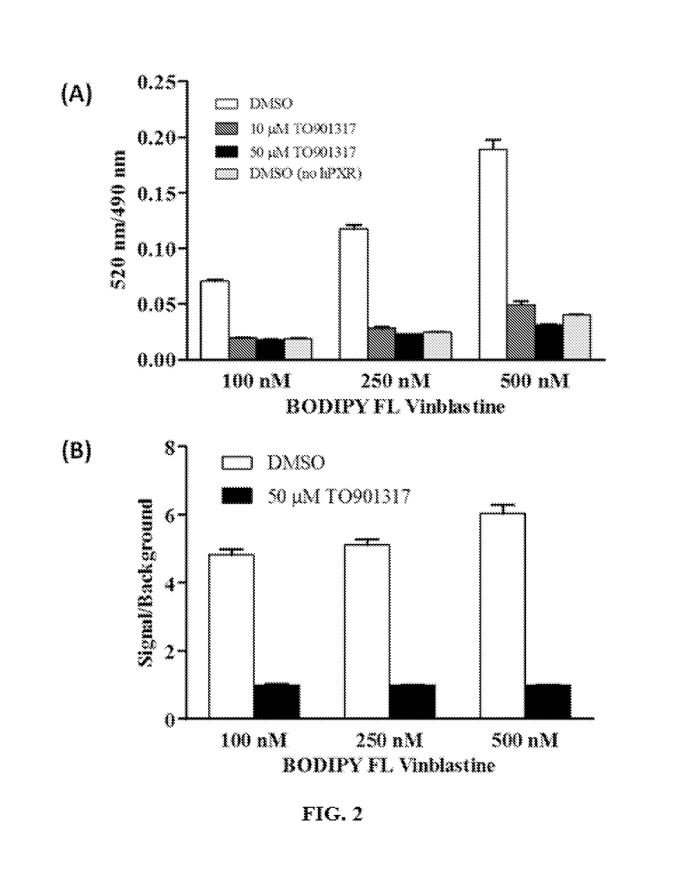

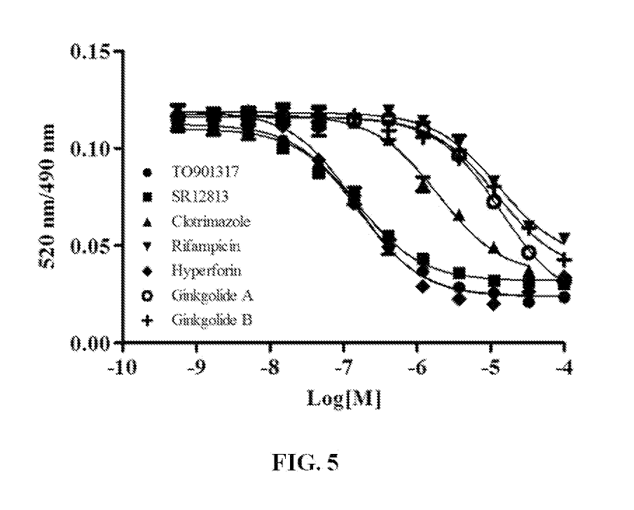

In a fluorescence-based assay, an optimal fluorescent probe concentration is crucial. To avoid deviating from the Cheng-Prusoff equation for subsequent calculation of a compound's Kᵢ value, a concentration at or somewhat below the probe's K_{d} value should generally be tested first (Cheng, Y. and Prusoff, W. H. Biochem. Pharmacol. 1973, 22 (23), 3099-3108). As the K_{d} value of BFLV is 673 nM, three different concentrations of BFLV were test: 100, 250, and 500 nM. Each probe concentration was tested under 4 different treatment conditions to gain insight about the total binding of BFLV to hPXR (DMSO group), the non-specific binding of BFLV under different concentrations of the competing ligand TO901317, and whether the non-specific binding is PXR-mediated (DMSO in the absence of hPXR protein) (FIG. 2A). Consistent with the data shown in FIG. 1B, in FIG. 2A, both total binding and non-specific binding increased when the concentration of BFLV increased. 10 μM and 50 μM TO901317 inhibited the TR-FRET signal equally effectively at a lower concentration of BFLV (100 nM), with fluorescent emission ratios of 0.019 and 0.018, respectively; however, 50 μM TO901317 inhibited the signal more effectively than 10 μM TO901317 at higher BFLV concentration (500 nM), with fluorescent emission ratios of 0.031 and 0.050, respectively. The positive control (TO901317) was tested at concentrations as high as 100 μM, but no additional inhibition of BFLV binding was observed beyond that achieved by 50 μM of TO901317 (data not shown; see the dose-responsive curves in FIG. 5A). Therefore, 50 μM TO901317 was used as the positive control in subsequent experiments to determine the assay background and noise signal levels.

To determine whether non-specific BFLV binding is mediated by hPXR, hPXR protein was omitted from the assay. In the absence of GST-hPXR-LBD, increasing BFLV concentration (100, 250, and 500 nM) correspondingly increased the fluorescent emission ratio (0.019, 0.025, and 0.04, respectively), suggesting that BFLV can bind weakly to other components of the assay system, such as the Tb-anti-GST antibody (FIG. 2A). BFLV binding in the absence of hPXR protein (fluorescent emission ratio 0.019, 0.025, and 0.04, corresponding to 100, 250, and 500 nM of BFLV) was comparable to the non-specific BFLV binding in the presence of GST-PXR-LBD protein and either 50 μM TO901317 (0.018, 0.023, and 0.031) or 10 μM TO901317 (0.019, 0.028, and 0.050). These results indicate that while BFLV can bind weakly and non-specifically to the assay system in an hPXR-independent manner, its binding to hPXR is specific and can be abolished by a higher concentration of the hPXR ligand TO901317. The ratio of total binding signal (DMSO negative control group) to non-specific binding signal (50 μM TO901317 positive control group) is shown in FIG. 2B as signal/background: 4.8, 5.1 and 6.0 for BFLV concentrations of 100, 250, and 500 nM, respectively. As the TR-FRET assay is a robust assay and is radiometric, all three signal/background ratios are suitable for a high-throughput screening (HTS) assay. In fact, with the Invitrogen PXR TR-FRET kit, HTS of 8280 chemicals was successfully accomplished, with signal/background ratios ranging from 2.5 to only 3.5 and Z′-factor>0.5; see Shukla, S. J., et al. Assay. Drug Dev. Technol. 2009, 7 (2), 143-169). In the further studies discussed herein, 250 nM BFLV was used in view of the both the signal/background ratio (the higher he better) and non-specific binding (the lower the better). However, 100 nM and 500 nM of BFLV probe concentrations can also be used.

11. Signal from the BODIPY FL Vinblastine-Based hPXR TR-FRET Assay is Stable

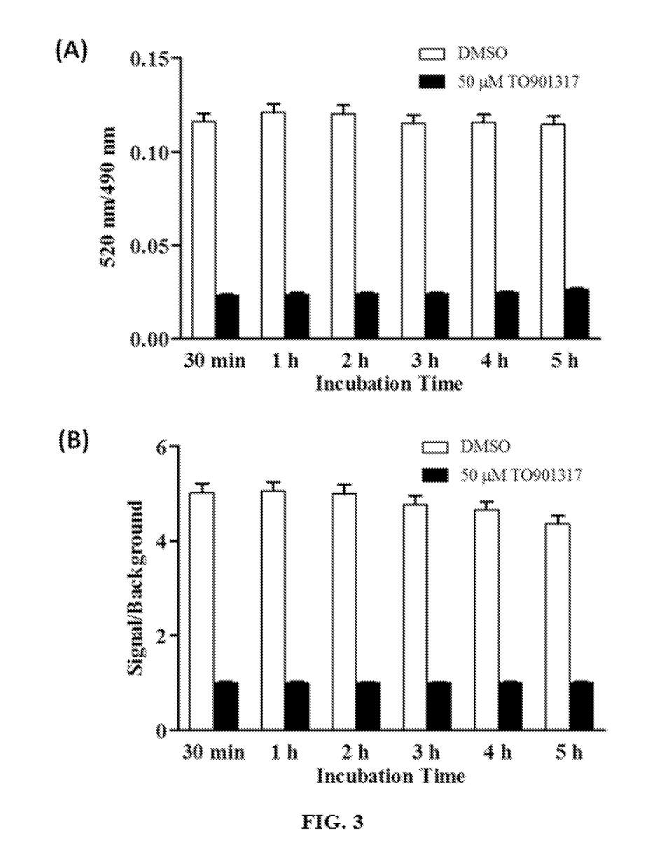

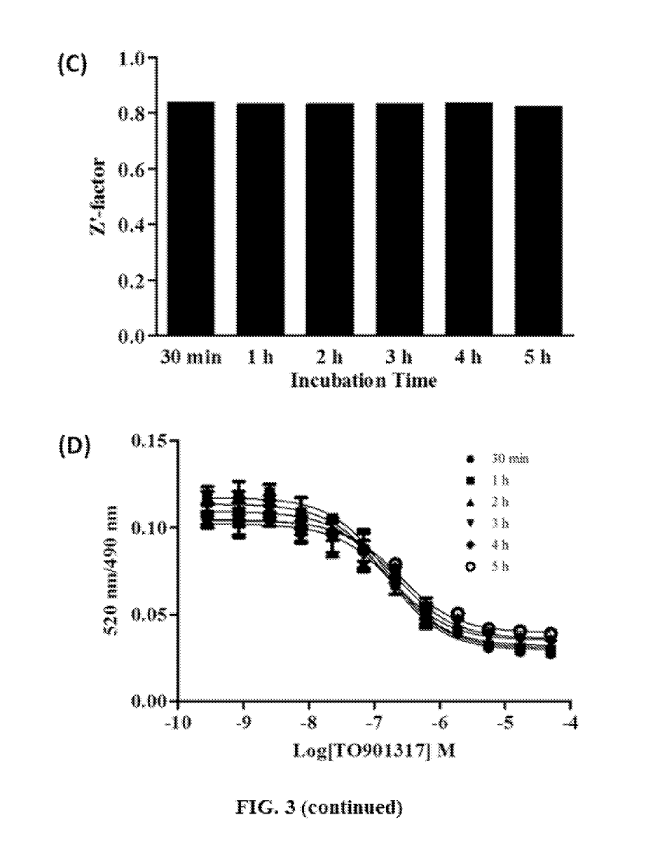

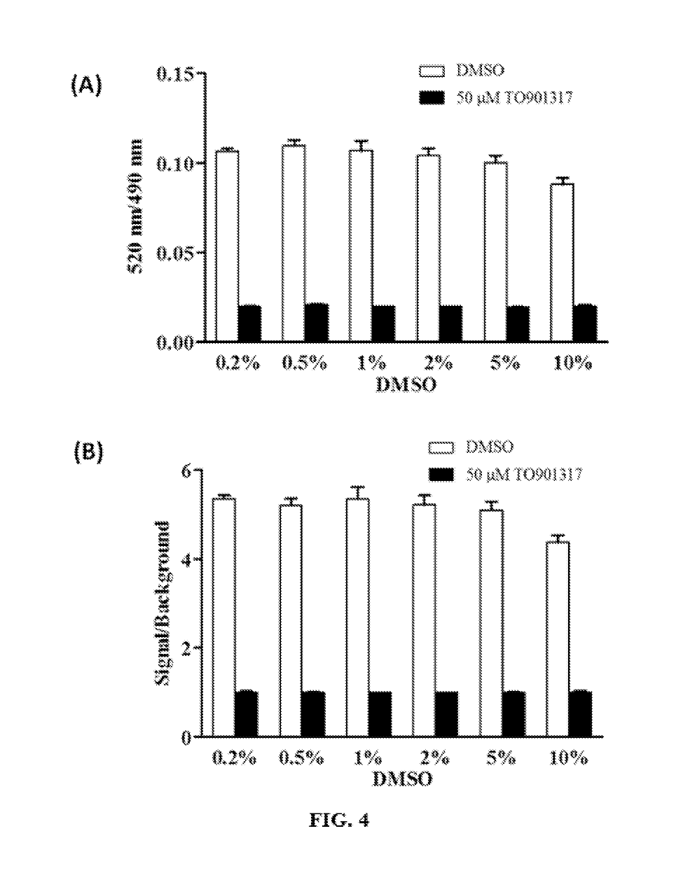

Signal stability is an important parameter in an HTS assay. To assess the signal stability, the binding activity of 250 nM BFLV was measured with or without various concentrations of TO901317 at 0.5, 1, 2, 3, 4, and 5 hours in reactions containing 5 nM GST-hPXR-LBD and 5 nM Tb-anti-GST. Both the total binding (with DMSO) and the non-specific binding (with 50 μM TO901317) were relatively stable (FIG. 3A). Correspondingly, the signal/background ratios were relatively stable, with a slightly decreasing trend over time: 5.02, 5.05, 4.99, 4.77, 4.66, and 4.36 for 0.5, 1, 2, 3, 4, and 5 h, respectively (FIG. 3B). The Z′-factor remained constant over the entire testing period: 0.840, 0.834, 0.831, 0.834, 0.836 and 8.835 at 0.5, 1, 2, 3, 4, and 5 hours, respectively (FIG. 3C). The IC₅₀ values for TO901317 increased slightly during the first 2 h (162 nM, 170 nM, and 185 nM at 0.5, 1, and 2 h, respectively) and then progressively increased to 231 nM, 266 nM and 299 nM after 3, 4 and 5 hours respectively, of incubation (FIG. 3D). The consistent Z′-factor values over 5 hours demonstrated that the assay is very stable and is suitable for HTS. While all incubation times can be used for HTS, the signal/background ratio and IC₅₀ values suggest an optimal incubation time of 2 h or less for the BFLV-based hPXR TR-FRET assay; an HTS assay using the Invitrogen PXR TR-FRET kit also demonstrated an optimal incubation time of approximately 2 hours (Shukla, S. J., et al. Assay. Drug Dev. Technol. 2009, 7 (2), 143-169.). Because a shorter incubation time contributes to higher throughput, a 0.5 h incubation time was selected for further experiments.

12. BODIPY FL Vinblastine-Based hPXR TR-FRET Assay Tolerates a Wide Range of DMSO Concentrations

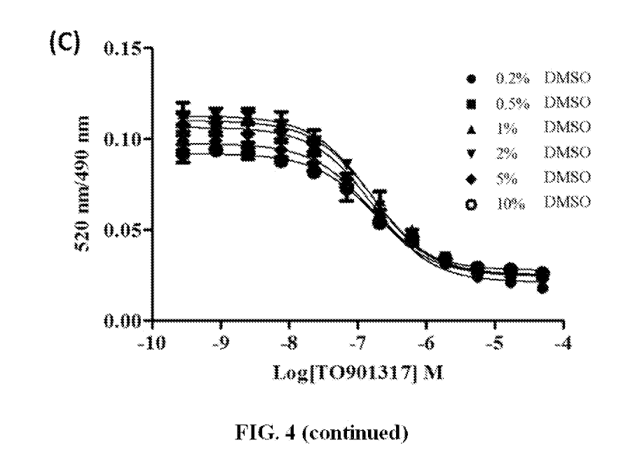

DMSO tolerance is another important assay parameter, as DMSO is a solvent commonly used for compounds in drug discovery. In the DMSO tolerance test, the TR-FRET signals from BFLV binding were collected after 30 minutes of incubation with either DMSO vehicle control or various concentrations of TO901317 in 0.2%, 0.5%, 1%, 2%, 5% and 10% final DMSO concentrations, plus 250 nM BFLV, 5 nM GST-hPXR-LBD, and 5 nM Tb-anti-GST. The total binding signal (at 520 nm/490 nm ratio) remained stable at 0.106, 0.109, 0.106, 0.104, and 0.100 when the DMSO concentration increased (0.2%, 0.5%, 1%, 2%, and 5%), and then decreased to 0.088 when the DMSO concentration increased to 10% (FIG. 4A). The non-specific binding signal (in the presence of 50 nM of TO901317) remained constant at 0.0199, 0.0210, 0.0200, 0.0199, 0.0196, and 0.0201, corresponding to DMSO concentrations of 0.2%, 0.5%, 1%, 2%, 5%, and 10% (FIG. 4A). The signal/background ratio was also relatively stable, ranging from 5.35 to 5.09 at DMSO concentrations below 5%, and falling to 4.36 when the DMSO concentration was 10% (FIG. 4B). In the TO901317 competition assay, the IC₅₀ values of TO901317 remained constant throughout the DMSO concentration range tested: 158, 160, 154, 164, 152, and 160 nM, corresponding to DMSO concentrations of 0.2%, 0.5%, 1%, 2%, 5%, and 10% (FIG. 4C). These results indicate that the BFLV-based TR-FRET assay can tolerate a wide range of DMSO concentrations up to 5%, comparable to the Invitrogen PXR TR-FRET kits (LanthaScreen TR-FRET PXR (SXR) Competitive Binding Assay Kit. http://tools.invitrogen.com/content/sfs/manuals/lanthascreen_{—}PXR_man.pdf, (accessed September 2012)). A DMSO concentration up to 1.1% was used in this report with the exception of the DMSO tolerance test.

13. Binding Affinity of a Panel of hPXR Ligands in the BODIPY FL Vinblastine-Based hPXR TR-FRET Assay

To further validate and evaluate the BFLV-based hPXR TR-FRET assay, a panel of seven hPXR ligands was evaluated: TO901317, SR12813, clotrimazole, rifampicin, hyperforin, ginkgolide A and ginkgolide B. The hPXR binding activity values of all seven compounds have previously been derived by using a commercially available hPXR TR-FRET binding kit from Invitrogen (Lin, W., et al., J. Biol. Chem. 2008, 283 (45), 30650-30657; Duniec-Dmuchowski, Z., et al., Drug Metab Dispos. 2009, 37 (4), 900-908; Shukla, S. J., et al. Assay. Drug Dev. Technol. 2009, 7 (2), 143-169; Lau, A. J., et al., J. Pharmacol. Exp. Ther. 2010, 335 (3), 771-780; Dong, H., et al., et al., BMC. Biochem. 2010, 11, 23; Chen, Y., et al. J. Pharmacol. Exp. Ther. 2010, 334 (3), 999-1008; and Hereley, S. B., et al., “Fluorescence-based Biochemical Assays for the Study of Pregnane X Receptor and Constitutive Androstane Receptor,” http://tools.invitrogen.com/content/sfs/posters/1007-ISSX-PXR-CAR-poster-.pdf, (accessed September 2012)). In addition, TO901317 (Xue, Y., et al. Bioorg. Med. Chem. 2007, 15 (5), 2156-2166), SR12813 (Watkins, R. E., et al., Science 2001, 292 (5525), 2329-2333; Watkins, R. E., et al., J. Mol. Biol. 2003, 331 (4), 815-828), rifampicin (Chrencik, J. E., et al. Mol. Endocrinol. 2005, 19 (5), 1125-1134), and hyperforin (Watkins, R. E., et al., Biochemistry 2003, 42 (6), 1430-1438) ligand-hPXR co-crystal structures have been reported. In the BFLV-based TR-FRET assay, TR-FRET signals were collected after incubation for 30 min with serial dilutions of the seven hPXR ligands, plus 250 nM BFLV, 5 nM GST-hPXR-LBD, and 5 nM Tb-anti-GST. The data were fit to a one-site competitive binding equation to derive the dose response curves (FIG. 5). Because of incomplete solubility, data points for clotrimazole 100 μM were not included. TO901317, SR12813, clotrimazole, rifampicin, hyperforin, ginkgolide A, and ginkgolide B had IC₅₀ values of 159 nM, 157 nM, 1.94 μM, 12.7 μM, 147 nM, 13.7 μM, and 12.1 μM, respectively (Kᵢ values of 116 nM, 114 nM, 1.41 μM, 9.26 μM, 107 nM, 9.99 μM, and 8.82 μM, respectively; see Table 1 below).

TABLE 1

| | Activity* | | | | Activity** | |
|---|---|---|---|---|---|---|
| | (BFLV probe) | | | | (Fluormone PXR (SXR) Green as Probe) | |
| Chemical | IC₅₀ | | Kᵢ | | IC₅₀^{†} | Kᵢ^{††} |
| TO901317 | 159 | nM | 116 | nM | 44 nM,^{a} 52 nM,^{b} 90 nM^{c} | 22 nM, 26 nM, 45 nM |
| SR12813 | 157 | nM | 114 | nM | 710 nM,^{c} 42 nM,^{d} 49 NM,^{e} | 21 nM, 24.5 nM, 34.5 nM, |
| | | | | | 69 nM,^{b} 140 nM^{f} | 70 nM, 355 nM |
| Clotrimazole | 1.94 | μM | 1.41 | μM | 2.0 μM^{b} | 1.0 μM |
| Rifampicin | 12.7 | μM | 9.26 | μM | 14.13 μM^{b} | 7.06 μM |
| Hyperforin | 147 | nM | 107 | nM | 140 nM,^{b} 710 nM^{c} | 70 nM, 355 nM |
| Ginkgolide A | 13.7 | μM | 9.99 | μM | 54% (1000 μM)^{g} | NA^{§} |
| Ginkgolide B | 12.1 | μM | 8.82 | μM | 48% (1000 μM)^{g} | NA |

*Activity determined using BFLV as a probe in a TR-FRET assay as described herein above.
**Activity determined using the commercially available Fluormone PXR (SXR) Green as a probe and as previously described in published reports (see references as indicated above).
^{†}Values obtained from published reports (see references as indicated above).
^{††}Values calculated from published data by using equation 2 (as described herein above).
^{§}“NA” indicates that the parameter is not applicable.
^{a}Duniec-Dmuchowski, Z., et al., Drug Metab Dispos. 2009, 37 (4), 900-908.
^{b}Hereley, S. B., et al., Fluorescence-based Biochemical Assays for the Study of Pregnane X Receptor and Constitutive Androstane Receptor. http://tools.invitrogen.com/content/sfs/posters/1007-ISSX-PXR-CAR-poster-.pdf, (accessed September 2012).
^{c}Shukla, S. J., et al., Assay. Drug Dev. Technol. 2009, 7 (2), 143-169.
^{d}Dong, H., et al., BMC. Biochem. 2010, 11, 23.
^{e}Lin, W., et al., J. Biol. Chem. 2008, 283 (45), 30650-30657.
^{f}Chen, Y., et al., J. Pharmacol. Exp. Ther. 2010, 334 (3), 999-1008.
^{g}Lau, A. J., et al., J. Pharmacol. Exp. Ther. 2010, 335 (3), 771-780.

Of the ligands with previously reported IC₅₀ values (TO901317, SR12813, clotrimazole, rifampicin, and hyperforin), the BFLV-based TR-FRET assay yielded IC₅₀ or Kᵢ values in the order (low to high) of hyperforin SR12813 TO901317<clotrimazole<rifampicin, in general agreement with the order reported using the Invitrogen hPXR binding kit (SR12813≈TO901317<hyperforin<clotrimazole<rifampicin), although the values measured with the BFLV-based TR-FRET assay were slightly higher (Table 1). The Kᵢ value of hyperforin was similar to those of TO901317 and SR12813 in the BFLV-based assay but higher than those of TO901317 and SR12813 obtained by using the Invitrogen kit (Table 1). The reported activity of SR12813 and hyperforin varies, possibly reflecting different assay conditions. The comparison data in Table I are the lowest reported IC₅₀ values. Both ginkgolide A and ginkgolide B are reported to be hPXR ligands, but their IC₅₀ or Kᵢ values had not been reported. In the BFLV-based TR-FRET assay, both ginkgolide A and ginkgolide B competed with BFLV for binding to hPXR in a dose-dependent manner (FIG. 5); similar curves were reported using Fluormone PXR (SXR) Green as the fluorescent probe (Lau, A. J., et al., J. Pharmacol. Exp. Ther. 2010, 335 (3), 771-780).

The results obtained with the assay system of the present invention indicate that BFLV binds to hPXR with high affinity and that the BFLV-based hPXR TR-FRET assay can be used to measure the binding affinity of compounds to hPXR. The rank order of affinity of hyperforin, R12813, and TO901317 determined by the inventive assay was slightly different from that determined by the Fluormone PXR (SXR) Green-based assay (Table 1). This discrepancy may reflect different binding modes of BFLV and Fluormone PXR (SXR) Green to hPXR. It is well known that PXR has a relatively large and flexible ligand-binding pocket that can accommodate ligands of different sizes (Xue, Y., et al., Bioorg. Med. Chem. 2007, 15 (5), 2156-2166; Watkins, R. E., et al., Science 2001, 292 (5525), 2329-2333; Watkins, R. E., et al., J. Mol. Biol. 2003, 331 (4), 815-828; Chrencik, J. E., et al., Mol. Endocrinol. 2005, 19 (5), 1125-1134; Watkins, R. E., et al., Biochemistry 2003, 42 (6), 1430-1438; Cheng, Y., et al., Protein Sci. 2011, 20 (10), 1713-1719; and Xue, Y., et al., Mol. Endocrinol. 2007, 21 (5), 1028-1038), and even a single ligand with different conformations (Watkins, R. E., et al., Science 2001, 292 (5525), 2329-2333). It would be of interest to use a co-crystal structural approach to compare the binding modes of BFLV-hPXR and Invitrogen's Fluormone PXR (SXR) Green-hPXR.

14. BODIPY FL Vinblastine is a Unique Chemical Entity with High Binding Affinity to hPXR

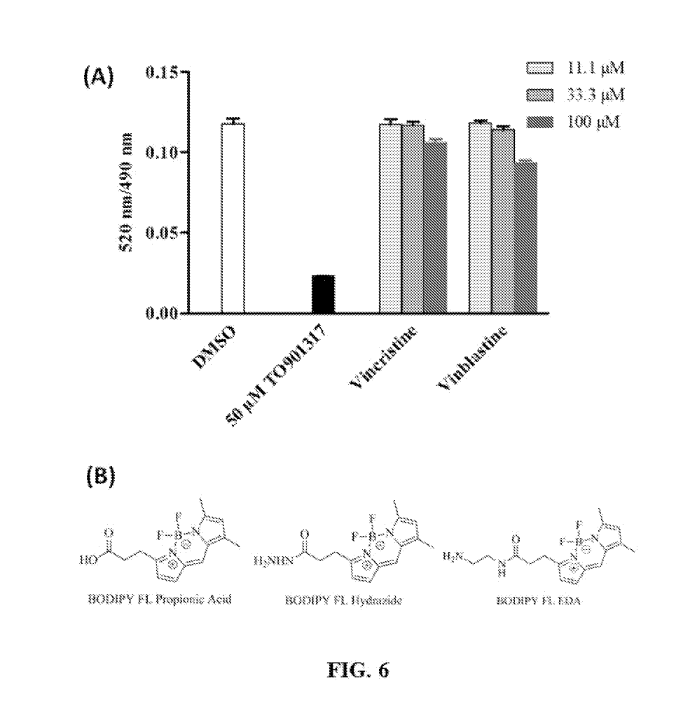

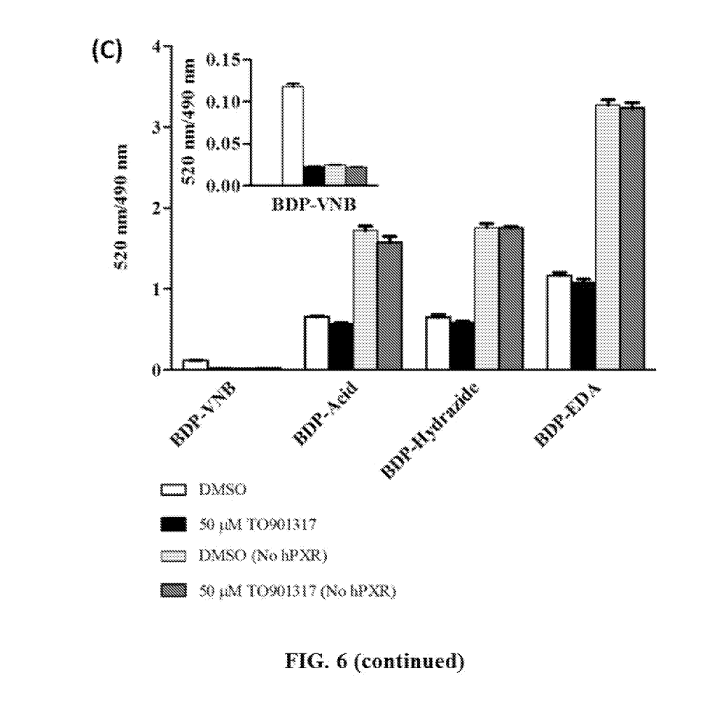

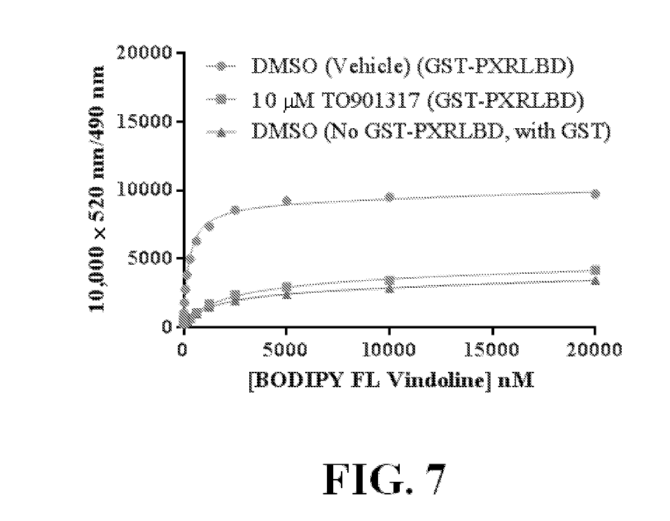

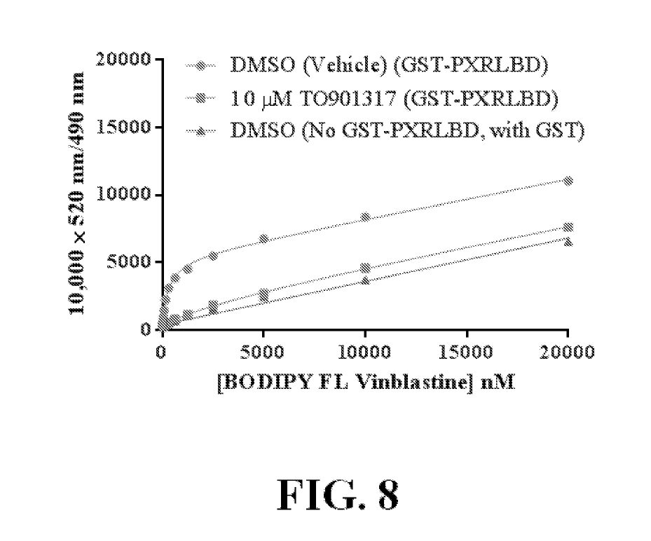

Because BFLV binds to the LBD of hPXR with high affinity (K_{d}=673 nM), the moieties critical to binding were investigated, e.g. was the high affinity due to BODIPY FL or to vinblastine. Vinblastine has been shown to activate hPXR in cell-based assays (Harmsen, S., et al., Cancer Chemother. Pharmacol. 2010, 66 (4), 765-771; Smith, N. F., et al., Ann. Pharmacother. 2010, 44 (11), 1709-1717). However, there is no evidence of direct interaction between vinblastine and hPXR. The effect of DMSO, TO901317, and vinblastine or its close analog, vincristine, on binding of BFLV to hPXR was assessed. Briefly, 250 nM BFLV was incubated with 5 nM GST-hPXR-LBD and 5 nM Tb-anti-GST for 30 minutes in the presence of DMSO, TO901317 (50 μM), and the indicated concentrations of vinblastine or its close analog, vincristine (see FIG. 6). As expected, 50 μM TO901317, but not DMSO, significantly decreased the binding of BFLV to hPXR (FIG. 6A). Surprisingly, vinblastine and vincristine at 11.1 and 33.3 μM failed to compete with and inhibit the binding of BFLV to hPXR. At 100 μM, vinblastine and vincristine only marginally inhibited the binding of BFLV to hPXR (25.4% and 12.1%, respectively) (FIG. 6A). Although the interaction was very weak, this is the first evidence that vinblastine directly binds to hPXR. The slightly higher binding affinity of vinblastine than of vincristine is consistent with cell-based assays in which vinblastine is a stronger activator of hPXR than vincristine (Harmsen, S., et al., Cancer Chemother. Pharmacol. 2010, 66 (4), 765-771).

The unexpected failure of vinblastine to efficiently compete with and inhibit the binding of BODIPY FL-labeled vinblastine (BFLV) to hPXR suggested that whether or not BODIPY FL fluorophore mediates the binding of BFLV to hPXR should be assessed. The binding affinity of 250 nM of BFLV, BODIPY FL propionic acid, BODIPY FL hydrazide and BODIPY FL EDA (FIG. 6B), in the presence of either DMSO (total binding) or 50 μM TO901317 (non-specific binding), was examined after incubation for 30 minutes with 5 nM Tb-anti-GST and 5 nM GST-hPXR-LBD (for hPXR mediated binding) or no GST-hPXR-LBD (for non-hPXR-mediated binding).

As shown in FIG. 6C, binding of BFLV to hPXR was specifically inhibited by 50 μM of TO901317. In contrast, BODIPY FL propionic acid, BODIPY FL hydrazide and BODIPY FL EDA generated high non-specific, hPXR-independent TR-FRET signals (1.72, 1.75 and 3.27 for BODIPY FL propionic acid, BODIPY FL hydrazide, and BODIPY FL EDA, respectively), possibly by interacting with the Tb-anti-GST antibody. In the presence of GST-hPXR-LBD, the TR-FRET signals decreased to 0.65, 0.65, and 1.16 for BODIPY FL propionic acid, BODIPY FL hydrazide and BODIPY FL EDA, respectively. The TR-FRET signals generated by BODIPY FL propionic acid, BODIPY FL hydrazide and BODIPY FL EDA were not inhibited by the hPXR-specific ligand TO901317 in either the presence or absence of GST-hPXR-LBD. These results demonstrate that the BODIPY fluorophore, in either its acid form (BODIPY FL propionic acid) or its basic form (BODIPY FL hydrazide and BODIPY FL EDA), can generate strong TR-FRET assay signals that are independent of hPXR, possibly by interacting non-specifically with the Tb-anti-GST antibody. Therefore, the BODIPY fluorophore does not bind to hPXR. Interestingly, BODIPY is reported to enhance ligand binding affinity to certain target proteins.

In summary, the data show that vinblastine labeled with the BODIPY FL fluorophore (BFLV) can bind to GST-hPXR-LBD in a TR-FRET assay containing Tb-anti-GST antibody. Binding can be inhibited by either high affinity hPXR ligand TO901317 (FIG. 1) or by omitting GST-hPXR-LBD from the assay (FIG. 2A), indicating that BFLV binds specifically to the LBD of hPXR in the assay system. The specific binding of BFLV to hPXR LBD in the assay system was confirmed by showing that a panel of hPXR ligands competed with and inhibited the binding of BFLV to hPXR LBD (FIG. 5 and Table 1). The data further demonstrate that BFLV is a unique chemical entity that binds with high-affinity (K_{d}=673 nM) to the LBD of hPXR and that differs from either vinblastine or the fluorophore BODIPY FL. First, vinblastine, even at 33.3 μM, failed to compete with and inhibit the binding of 250 nM of BFLV to hPXR LBD (FIG. 6A). Although vinblastine showed weak binding activity at 100 μM, the receptor binding affinity of vinblastine is not at all comparable to that of BFLV. Second, although both acid and basic forms of the BODIPY FL fluorophore generated high TR-FRET assay signals, the signal was not dependent on the presence of hPXR; therefore, hPXR LBD does not interact with the BODIPY FL fluorophore (FIG. 6C). These data indicate that BFLV is a high-affinity hPXR ligand. The BFLV-based hPXR TR-FRET assay has a high signal/background ratio (FIG. 2B) and high signal stability, both of which contribute to high and consistent Z′ values (FIG. 3); it also has a wide range of DMSO tolerance (FIG. 4). In testing a panel of hPXR ligands, this validated assay produced results comparable to those of Invitrogen's hPXR TR-FRET assay, whose probe [Fluormone PXR (SXR) Green] has an undisclosed structure.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A composition comprising a polypeptide comprising a pregnane X receptor polypeptide, or a ligand binding fragment polypeptide thereof; and a molecule comprising a BODIPY residue and a vinca alkaloid residue.

2. The composition of claim 1, wherein the polypeptide comprises pregnane X receptor polypeptide.

3. The composition of claim 1, wherein the polypeptide comprises a pregnane X receptor ligand binding fragment.

4. The composition of claim 1, wherein the pregnane X receptor ligand binding fragment comprises amino acids about 130 to about 473 of human pregnane X receptor, or the homologous amino acids in a non-human pregnane X receptor.

5. The composition of claim 1, wherein the pregnane X receptor polypeptide is human pregnane X receptor polypeptide.

6. The composition of claim 1, wherein the pregnane X receptor polypeptide further comprises a heterologous polypeptide.

7. The composition of claim 6, wherein the heterologous polypeptide comprises one or more affinity tags or epitope tags.

8. The composition of claim 1, wherein the molecule comprising a BODIPY residue and a vinca alkaloid residue is:

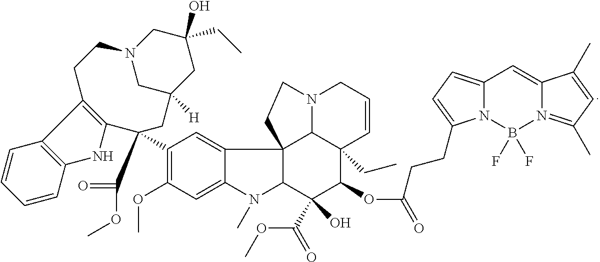

9. A method of identifying a test compound that binds to pregnane X receptor, the method comprising the steps of:
a) providing a solution comprising:
i) a test compound;
ii) a chimeric polypeptide comprising a pregnane X receptor polypeptide, or a ligand binding fragment polypeptide thereof, and a heterologous polypeptide;
iii) a fluorescent donor molecule comprising chelated terbium and one or more moieties capable of binding the heterologous polypeptide; and
iv) a fluorescent acceptor molecule comprising a BODIPY residue and a vinca alkaloid residue;
b) illuminating the solution, thereby causing fluorescence in the fluorescent donor molecule; and
c) measuring fluorescence emission from the fluorescent donor molecule and the fluorescent acceptor molecule.

10. The method of claim 9, wherein the illuminating is at about 340 nm.

11. The method of claim 9, wherein the measuring fluorescence emission from the fluorescent donor molecule is at about 490 nm.

12. The method of claim 9, wherein the measuring fluorescence emission from the fluorescent acceptor molecule is at about 520 nm.

13. The method of claim 9, wherein the polypeptide comprises a pregnane X receptor ligand binding fragment.

14. The method of claim 9, wherein pregnane X receptor ligand binding fragment comprises amino acids about 130 to about 473 of human pregnane X receptor of the 473 amino acid isoform, amino acids about 141 to about 434 of human pregnane X receptor of the 434 amino acid isoform, or the homologous amino acids in a non-human pregnane X receptor.

15. The method of claim 9, wherein the pregnane X receptor polypeptide is human pregnane X receptor polypeptide.

16. The method of claim 9, wherein the heterologous polypeptide comprises one or more affinity tags or epitope tags.

17. The method of claim 9, wherein the molecule comprising a BODIPY residue and a vinca alkaloid residue is:

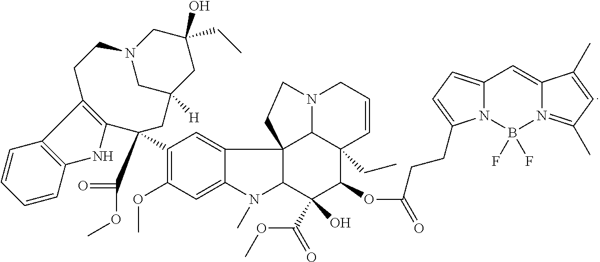

18. The method of claim 9, wherein the chelated terbium comprises at least one diethylenetriaminepentaacetic acid residue and Tb⁺³.

19. The method of claim 9, wherein the one or more moieties capable of binding the heterologous polypeptide comprise an antibody.

20. A kit for measuring the binding activity of a test compound to a pregnane X receptor polypeptide, comprising:
a) a chimeric polypeptide comprising a pregnane X receptor polypeptide, or a ligand binding fragment polypeptide thereof, and a heterologous polypeptide;
b) a fluorescent donor molecule comprising chelated terbium and one or more moieties capable of binding the heterologous polypeptide; and
c) a fluorescent acceptor molecule comprising a BODIPY residue and a vinca alkaloid residue;
and optionally, one or more of:
d) a compound known to bind pregnane X receptor;
e) instructions for measuring a fluorescence emission ratio of the fluorescent acceptor molecule to the fluorescent donor molecule in the presence of a test compound; and
instructions for determining the Kᵢ or IC₅₀ of a test compound from a fluorescence emission ratio of the fluorescent acceptor molecule to the fluorescent donor molecule in the presence of a test compound
